SEQUENCE LISTING (33 sequences)

SEQ ID NO: 1 - DNA, 32329 bp, Streptomyces cattleya
  ref <301>: AUTHORS: Nunez, Luz Elena Mendez, Carmen Brana, Alfredo F. Blanco, Gloria Salas, Jose A.
  ref <302>: TITLE: The Biosynthetic Gene Cluster for the ??Lactam Carbapenem Thienamycin in Streptomyces cattleya
  ref <303>: JOURNAL: Chemistry and Biology
  ref <304>: VOLUME: 10
  ref <306>: PAGES: 301-311
  ref <307>: DATE: 2003-04-21
  ref <308>: DATABASE ACCESSION NUMBER: AJ421798
  ref <309>: DATABASE ENTRY DATE: 2001-12-11
ggatccgcgcggcggcccgggcgggcacctcggtgcgccaggcacgggcgatctccggccgcgcgagcaccgccttgagcagggcgtacagcggtgagcgcggcggggcggcgcgcaggtcgtcggagatctcgcacagcagcggcaccagccacccctcgtaccagtcggccagcagccgcggggcgtcggggtggaggcagatgaatcgggccaggttctcccggggtatggcctcgggcacccgggcgacgccggagaagagccgggtgtaggcggcgttgcaggccagcagccgccaggcggggtcgcgcaggtaggcgggcatcccgacgtcccggaagccgcggtgccattcgcgggcgaagccggccgggtcgccggggtgcggggtctcgggggcgggggccgggcgggcggcgggggtacgggaaccggccagcccggccagttcccgctcctcctcgcgggtgagggcgagggcggcggcgaggtcggcgacgcggcgcgggtccacgccgtgctgtccgcgttcccagttgctgaccgtgcgtgtgctgacgtccagccgccaggccagcccttcctggctgagtcctgagcgcagtcggcgggcgcgcaggaacgccgggagcgggtggccggccgacggcccgccgggcggggcggcgcggtcggggcggcggtcggccggccggtgccgcgctccgccgccgcggtcgccgagggcggggaacgaacgtggaatcatgtgcggtacgccttgtcgaagccgatcggacggtgacgggtcacggacggtcacggaggaggtggccggggcgcgacctggcgccggcccccgtcacggacgtggcgccgtgctgtcgccgccgtcggtgaccaggccggactcccccggctcgacctggtacgggcaccgattgtggccgttgtggcgggggttggggagagaagcgatttccacggacgtccgatccctattattcacctttcaccaaccggcccgccggtttctgttctggatggagacatgaccgacgcgcaaccttccggccccgtcgtggagcacggacgcggagacctcaccgccgaggagcggcgccagaagctgtccttcctgcgcgagaagcgcgacagcgtcgaccggtcctccttcgaggcccgctaccccgtcgaggtgccacggcggctccccggccggggccgccacgcccccgggctcaccctggtcgagatgggccgcctgctcggcctgcgggacggctcgtggtacgcggacctggagcgcggccaggtggacaacgccccggtggaccggctgttcaaggtcgccgagctactggacctgacgcctcgtcagtacgagttcctgtgcgtgtacgcctgccggcccaagcccggttacccggcggccgggctccccggcggctaccgggaggtgctcgacctgtaccaggggccggcgtacttccaggacgccgggtggaacctgctcaggccgaagatgtacaacgcggcggccgaagcgctcttcaacggcatccccgacgacttcaacttcatccgctgggtgctgctggaacggtcggtgcgccggccccccgagggcggccggcgccacgcctggctgccggacttctggcaggtgtgggcgccgcgcgcgctgccggtggtacgggccgcgtacgtcgcccggccggacaacgccaccctcgcccggctccacgccgacctggcggccgaccccgagatcggccccatctaccagggccgtctgccgccctacccgcacaccgacggcgacctgcgtccgttcgtccacggcagcaccggggaacgcggcatggtgcggatcaacgtcctcaccccgcgcggcagcgacgacaccctgatgctgctcaactggcaccccggcgcctgacaccgggcacatccctcagacggtcttgagcgcgccgccgtcgatcacgtagtcggcgccgtggacgctggccgccaggtcggacccgaggaaggcgatcaggtcggccacctcgcccggttcgaccagccggccggtcaactggccggagcgggcgggcactcgggccacgtagtcggccagctcctggccgccgagggcggcctgctgggcgccgtagccgtccgggtcctcccacaccggggtgcgggtgggccccggcgagacggtgttgacccggaccccgcgcggcccgaactcggcggcgagcgccttgcccagggtgttgagcgcggccttggcggtggcgtagtcgatcggcccggtggcgttgcgggcgccgatcgaggagacgttgacgatcgcgccgcgccgctcgatcagggagggcagggcggcccggatcaggcgtaccggggcgaagaagttgagcgcccaggagcgttcccagatctcgtcgtcggcggtgaggaacccggccgccgtgaagtggtcgccgccaccggcgttgttgaccagcaggtcgatcccgcccagttcggccagggcacgctggaccagctccgcgcagccctcgggggtgctgaggtcgaccgccaccggaaccgcgccggcgtccttcaactccgcggtgacggtccgcgcggcgcccaccacccggaccccctcgccggtcagcgtccgtaccaccgccagcccgatcccccggctcgcaccggtcaccacagcggtcttccccgacatccgcaggtccatcgtgtcctctcggtagcaactgctcccgccacgctatacgcacccgggccgcgtcccggtcggcgcgcggtggccggtaaccgccgttcatgcaagggggtttcatgatgccgcccggacgcgacgtgcctcagccgcgccggggcgtggcggccaccgcccgggcggcgccggggccttgggcccgggcggtgacgcgggcggaggccaggcggatcgcggtttcggtgcggtcgtcgacggcggggacgccggccgcggcggcgaaggcgccggaggggtcgtggaccacgcggagcgggggccgggccgacgctgggaaggcctggcgcaggcagccgcagagttcggcgacggacagccgggcgagggcgccgagttcgccggggtcggcggtgacgagtacggcggtgaccggggtgtccggcggcgcggcgcgtaccgcatcctcggcggcctccagccggtgcagtccgaggacggtgaccacctggtccggttcgccgtccgccgggagggggtcgccggtgaccgcgtcggtcgcgggcggcggggaccacggttcgtcgacgtggtggacgggcggcaggccgggcgcgggcggcgggtcgtcgtccagtggcaggccggccaggcgctcgcagacccgtacggcgtcgagcgggtcgaggaaaccgggcgcggcggcggccagttgggcggtgccgtgcagtgcggtgaggacggcctcggcgacgcggaccatgcggcgggcggggcgtccggcgggctccagccgttccagcgcgaggccgagcaggatcgcgtcgagcttcagcagctgggcgaagggccggcgggtggcggcgtcggtgatcacctcgggcatcaggtccatggcgagccgggccgggccggggccgtcgtccccggcgggcggcagccgcgagacccaggcccgggcgaacgcggccagggcgtcgccgccggagcgcaccggtcgcggcggtggtccctccggcacgcgttcggcaagcgcggccagcacggcgaagtagagcgcccgcttgccggggaagttggagtagaccgcaccacgggtgagcccggcgcgctcggcgatggcgtcgatcctggcgtcccggtagccgcgttcggcgaactcctcgcgggcggcgtccaacaccttgtcgcggttgcgttcctgggtctccgccctggtcagccgggccatcgtcctcctcgccacggggccgggcacggtcggccacggccggttcgccggtgacggccggcgccgggaaagcgcttgccacgcgcccctggtcaccatatcgccgcgttccccgcgcacgcggggcggccgggggcgcgggtggcggcaatcccgtggacaccgtgcgctcgcgcgccccacgatggagcccatgctcaccaccgcactcgcgttcctcggcgcctgcgtgctgatcgccgccgcgcccggtcccagcacgatgctcatcatccgccagtcgctgcacagcagacgggccggtttcctgaccgttctgggcaacgagaccggggtgctcacctggggcgtggtggcggcgctcgggctgaccgcgctgctggccgcctcccgcacggcgtacgacgtgatgcggatcggcggggcggtggtgctggtgtggtacggcgtgcagacgctgcgggccgcgcggcgcggcgaggcacggccctccgccgccgacgacgaggcggcggtggtgccccgttccggctggaagatctaccggtccgggctgctgctcaacctcgccaaccccaaggcggccgtcttcgccatgtccttcctgccgcagttcgtcccggccggtgcgcccaagctgccggtgatcaccgcgctcgccgcgttccaggcgctcttcgaggtcgggtactacgggatgtacgtgtggttcgtcggccggatgaagagggtgatctcccgggcgggcgtgcggcgccggctggagcaggtgtcggggggcgtgctggtgctgctgggcatccggatggcggtcgagagctgacgtagcgtcagctacccggtaggcccggcgcgtgcggcgcgtcacggatccgtgcggcgccgcccggcccggcgttcgcccccgccgccgccccgaacaccaccacaccaccggacccgtaccgctgaccgggtaccggcggcgggcataccgggacgggggacgagggctaccgaggcgggtgacggcgtgacgcttcccaccgagacctgggcggtgcggatccaccggcacggcgggccggaggtgctcgtccacgaacggctgccgttgccgccgctcggcccggccgacgtcctggtcgccgtcgacaccgcctcggtctccggctgggacgtgaagtaccggcgcggactgccgccgggtgcgcggctgcccggccgggaacggtaccggctgccgctgcaactcggccgcgaggcggcggggacggtgctcgccaccgggccggaggcggccggccgcttccgccccggagaccgggtggtcgccgtcgtccacccggagaacccgcgcgccccggagaccgtacgcggcctgggaaacctctccaccggcatcgccctccccggccaccaggcgccgggcggctacgcacgctacctgatctgccatcaggacatgtggctgcccttgccgtccggcgtcgacctcgaacaggccgccgtgacgctgtggccgtacgcgacctgccaccgcgtactgcgcgaccggttgcgggtggcgctcggcgagacgctgctggtgtgcggcgccaccggggcgatggggctggcggcgctgcgactggcccggctgaccggggtgcgggtgatcgcgatgacccgctaccgggccaaggagcgggcgttgcggacggccggcgcggacgaggtggtggtcgccggtgatccgcggcaggcgtgcgaggcggtgcgcgcgctcaccggcggggaaggcgtggaccacgcggtggacttcaccggcagcgccgcgctgctgcggctggccgtcgactcgctgcgcctcggcggccggctgtgcccggccgccacccagcgtccgcccggcccgctcccggtgaccaccggtgacctcacccggctggagatcaccgtgtacggcatccgcggcgcccgccaccgggacgcgctgcgggtgctcgccctgctgggcgacggcagcctgccggccaccccgatcgccgcccgcttcccgctgtcccgggccggcgccgcccacgagttcctggaacacaacaccacggctgtcggccgcgtggtgctcaaacccggctggtgaccgacgcgacccgccgccggccgccccgggcggcgtaccggcccgccggattccgggcgggacctcgggcgtcggccgggaatatgcgcgtgatcagcgttgactgccctgtgcacgctacctaggctctgctcagaacacagcacccgggcaccggcgcggacagcggcccttcgacgacacgacacgactggcgccgggcgcacggcagttggtccccggcggagcggacggaacggcgaatcggcgcgatgggcgcggccgccggcgagcggagaaccctctcacggaaaagaggcacgaccgtggatgtcccagtgaagcaggagtcgcggcaggaccggttcgaggatcaggacgaggaccggttcgcggcgcaggagtccatcgagtgctcgcggctcggcgacgggatcgccctggcggagttctcgggggcgcacgagcagaacccgttcagccgcgcgcggatgcgcgaactcaccgcgttgatgcgggagttggacgcggacgagaaggtcaggtgcgtggtgctgtacggcggcgccgggcggtcgttcggggtggggggcgacttccacgaggtgtcggagttcaccggcggggacgaggtcaacgcgtggatcgacgacatcaccgacctgtacaccacggtggcggcaatctccaagccggtgatcgccgccatcgacggttatgccatcggtgtcgggttgcagatctcgttgtgttgtgactaccggctcggcagtgagcaggcacggctggtgatgccggagttccgggtgggcatcgcctgcaacttcggcggtttcatgctggaggccgcggccggccgcaccgtgatgcagcgcatgctgctgacctgcgacgagtggccggccgaacgggcgctggcggacgggctgctgcacgagacggtggcctcgccccggctgctggaccgggccctggagctggcccggaccatctccggctacaccgccgaggccgtgcagtccacccgtccgcgggtcaacgcgcctttcgtggccgggctggagcggatccgccgcgaggcgaaggagtcgcaccgcagagccttcgcggccggcgaggcccaggtgcggatgcgccgcgtcatcgggcggagctgacctccccggcgggcgcgggggtcgcggtcacccggtacgcgtagtcgtagaaccgcgactcctcgcgccacccggtcgcccggtacagggcgagcccggcccggttgccggtgtccacggtgagcaccatctcgccacggccgcgggcccgggccgcggccatcaccgaccgcagcaggaaggcaccgaggcgccgaccgcgcatccgcggcagcacgccgatctgccgcaggtaggcggcgccccgggtctgcgccatgcggtcggtgacgttggcgaaccccaccggctcgccgtcgagttcgagcagcgcgcgctgggacggatcggcccccgggtcggcgccggtccagcgcagccactcctcgaagccgtccggctccatgccggcctcctcggcggagaaggcacggttcttcaccgcgtggaaggcccgcagccccgcctcaccctccggcacccgcaccaccgccccgggcaccgggggctcccccgccgtcgccccgcccagcggcaccgacatccgcaggtacggacgcggggcggcgaacccgtaggcgctgagcgcgatccgcagcgcggtctcccgttccgacacgaaacacgacagacacacctcgccttcgggccgcccggccgcgatggtccggcagcggcggtgggcgaagtcgagcagtgaggcggcgtcgtcgggggtggcgtgcggggcgagcaggagttcggcgtcccaggcggagcgccccgaggagcggctgagccacaccgcgccggccacctgtccgtcggagccggtgagcagcgcggtgtcgtgttccgggtcgagccccggggcggccagccggccggcgatccaggtggcaccgacctccacccggccgagcgcggcgcggtcgcggtcggcggccagccggaccagggcgggcagatggtctgcggggcgtatcgggaccagccgccgtgggctcaccgtccgctctccggcccggcggggtggcggggcagcgaatagcagtaggagacggcacgccggtcgtcctcggtggtgttgggcgccgaggtgtgggcgacgtagccgttgaacagcagcgccgagccggccttgcggggcagggtgaccggggtgaggccgggccagatgcgtgcgatgtccagggacggttcgcgcggtgaggcgaagatgtcgccggggtcgatgccgtccggttcgtagagtttgcgttcccggtgggagccgggcaggaagcgcaggcagccgttgtcctcgcgggcgtcgtccaccgcgatccagatggtgtagacgtccacgtacttctcgtgccactcgtcggccaggaagatggcgtcctggtgccagggtttctgggagccgacccgggagggcttggcccacaggaagcagtcgtagaagtcaccgggcaggccgtgcagcggttcgatcagcttctcgttgaggccgacgaggtcggggacggcggcgaggtcggcggagtgttcgtgggcgcggccgatgatgcgcaccttgccgggtatgcactccttgccggccgcccgggcggcccatccgccgccctccccctcggcctccagctgccattcctcggtgctgatgcgcatccgctcggtgcggccgatcagttcggcgcagccgcggcgcatcacctcgagtacggattcgggcaccaggtcgtccagcgccacgacaccgtcgcgccgcagttcgtcggacagggcctggatcgactgggtcaccggcatgggttccacctcttccggagaacacaaggccagccgtgctgccttggtagatccctgatcgccacaactatccggtggcggccgagggggaggcaacgcgccgcccgcagatgagtcgggggtcggccaaaaaggcatagcgcgcggcgctgccgtccgggccggtctcacttacgctgatgacccgtcatgtggggacaaggaccgacccgggaaccgggcggcgacaaccggggtgtgccgatggtgtgtgcagcgtccagtccggcgggcgccctgctggtcgactggggcggtgtgctgacccagccgttctacgccgggatcgccgagtgggccgcgcgggacggggtggacgccgacgccttccacgcgttgctggcccggcacctcggtccgggtgcgcccggtggcgcggcggcctccgtcttccaccgggtggagcgcggtgaggtgccggtggccgagctggaggtgacgctggcggagtcgctgcgccgcccggacggcaccgggccgcccgccgaggggctgatccagcgcatgttccagccgttcaccatggccggcgccatggtggagctggtgcgccgggtgcgcgcctcgggggcggcggtggcgttgctctccaactcctggggccacacctacgaccgcaccggctgggacggcctcttcgacgaggtggtcatctcctgcgaggtggggatgcgcaagccggagccggagatctaccggtacaccgcgcggcggctcggggtggcgccgcggcggtgtgtcttcctggacgatctgggacgcaacgtgcgggccgcggcggcggtggggatgaccgccgttcagcacacctcggtggaggagagcagcagggagctggcccggttcttcgacgtctcgccgctgccggccgggcgttgacggggcaggggtggcgtggtgctcgccgcggcccggcgcagcgcggcgacgagggtgtcgagccggtcctcggcggcctcggtgcagtgcagcagccggcgggccatgggggaaaggcaggtgccgcggggtgcgctgtgcaccagggtgcgtccgcagacctgttccaggcggtggatgcgccgggtgagggcgggttgggtgatcagcaggtcggacgcggcgcggttgaggctgccggtgcgttcgatgacccgcagcaggtagaggtgttccgcctcgaccagcgaggaggggccctcggccgtccagtggcgttcgcagaccaggaacggcagcgggtcgcgggggccgtcgggctcggtggcggcggaccggccaggcggcggcgccgacaggtcagcggcgggtaacgggaaggtggtggagcgcagatagccggggttgcgttcggcagcgtgctggacgtagcgccgctgcagcccggtacacaggtcacgggcgagcgcctcgggcagcagcagcggatcggtggcgaggaagaggcggcgcacgacctgctggcgcagcgggcgcagcgcgtacccggcgtcctcgaccggggccgcgcacagcggggagaccagggcgaagccgtagccgcgtgccaccaggtcggcggcggcgctgcgggagtggacggtgcagccgacgcgtggggtgagcccgtgccgggcgaaggcacgggccaggtcctcgtgggtggactcgcccacgatggcgacccattcggcctcggcgaggtcctccaggccgagggcgccgcggccggcgaggggatggccgcggggcaccgccacccattgcggctcctcgacgaccaggcgcacgtcggcgccgtagcgtcgcagcgccggggtggagcgggcccaaccgcaggcggcgtccacggcgtagcggtcgaagcgcgaccacacgtcctggcgggcggcgggcaccaggtcgaagacgacgtcggggaagccgaccgccggctcctcaccggcctcgcaggtgtattcggtggagacggccatctgcaggacgcgcagtcgcgggccgggttcgtcggtgtcggcggcgttggccatggcgaagtcgatggcgccgagcaggcgttggccggcgtgcagcagggcgtggccctcgtcggtgagccgggcgccggtggagccccgggagaccaggacggtacgcagccggcgttccagccgggtgaggcggcggctggcggacgactgggtgacccagccctggcgttgtgcggcgtgcagacttccgtggtcggctacggcttgcaacagtgctacatccgaacgtctcagatcgaacattcgctgctcctgtgcgctgtggtgccggcttcgggcggtgggcgaggaggggggaagcgctcgcacagttgtcgggtccgtaaccttcccgtcacgtcagccggtcaaccgtgcccgcggtgggggggttggtgccggtggtgtgcccgttcgagcgggcggcgcgggggaaggcggaaccggcgcagatcccgagcgcgccgagcaccgcggtgccggcgaagacgagcgtgtaggcggcggggctcggatggtcgccggtggtgccggcggcgagcaccatggagccggcctggacgccgaaggagccgccgaggctgcgcagcaggctgttggtgccgaccaccaccccggtggtgccgcccggcgaggccagggtgatcagctcggtcatcgcgccgccgaggacaccggtggccgcgccgttgagcagcgtgcccagggcgagggaggccatgctgaccggcaggaaggcgaagaccacggcggaggcggcgaagagcacggtgccggtgaccatcacggcccggggcggcagcacgcggcccagccgtccggacagcagcccgccgcccagcgtgccgatctccatcggcatcatcaccacgatcacaccgagcccggccaccccgaggccgccggccgagcgcggagcctcggcgatggccggcgggaacgtcccggtcacgaacatcaccaggccggtgaggaggctgaccgcgttgagtgtccagatcggccggcggctcatcagccgcaggtccatcatcggcgtggtggtgcgccgttcccaggcgagcagcgcggccccgctcaccacggcgccggccagcagcgtactgccggtgaccgagccgatcccgtcgcgcccgtatctgcccagcgccagcagcaccagggcgctgctgacggcgagcagggtgagcccgggggcgtcgaggcgctggccggcccggcgcggggtctccgggagcaccgccgcgccgatgagcagcagcagcgcggccagcgccgcgccggtggcgaacacccagcggtagccgtccgggtcgccctccaccaccccggccagcatgatgccggcgccgccgccgaccccgaacgacgcggagagcaccccgaacgggccggccagcgcacccgccgggaacccctcccgcaccaggccgaccgcgagcgggaagaggccgccggccacgcccatcaccagccgagcggccagcagcagcccgaaggtgtcggcgctcgcggccaccgcgccgccggccgcgaagacggcgagcaccgccagcagtacccggcgcctgccgtacaggtcgcccaggcgccccaccaccggggtggccacgcacgccaccagcaggtggccggagaccacccagccggtgtccgccgcgccgacgccgaagccgtgccgcaactgggtgagcagcatcgtcacggccagttgcatgagcgcgaacagcccgcaggcgctggccagtacggccagcgccacggtgctcgcgccgcgcgccggggagccgtgcgccggtgcgctcaccgctgctcggtcaggacgggctcggccggcgaggtgacctcggcggcttcgaaccggtgccggtagtggccgatgcgggagaccgggtagctggggccgatggcggcgcgggcgaacgccttgaggtcgccgggtaccggggtgtagcggccgttggcgccgaagcgctggtcggccaggcgcatccggaccgggcgcctgcgcagctcgccggtggtgaagtagccgggccagtcggcggtgtactcggtggtgcggccggccgccgggtcgtagtcggggcgcagcatcacgtcctgctggaaccggaagacctcggcgaggtcggggtcgctggtgtcgtccacgccgagggcggccacgaagtcacgcagttcggtgtaggtgcgttcgaagtcgttggcgatccgcagccagccccagttgtcgatggtccagccgcgccgcttgccgtaccgggcgaggtcggcggccatgtcgggctggctggcgaccaggttggccagcggcagttcggggatgtcgatgtagtcgtggtacagcttgcgcatccgcaggtagatcgagccgagcacggtgtcggggcgggcgccgaagtactgctggagccgttcgtagaaggcggtgtagccgatgtcgtggcgttgccgcaggtacatcgacagatagcgggtgtagcagccgttgtgcaggaactgcacggcctggacgaagccgaagcagtcgacccattcgtcccgggacatggcgttggtctccatcaccatgtccacggtctccgcctcgtcgtccggggtgccgggctcttcgacgtagagccgcttgggtacggttcgcaggccgtaaccggcgatcttctcgggggtgttgatgggggcgttgggcaggatgccgaactcgtagacgcggatgtcctcgtggttgccggcttccagcagggagccgatgccgtcccggaagctgcgcggtgactccagcgggaggccgaggatgagttcggtgtaggtgtggatgttctcggtggcgtaccgctgctggagtttgcggtagttctccaggccgatgttcttgcggtcgatggcctccagcacgtcgaggtcggtggactgcatggacaaggtggtgcccatcagcaggtcggcgtcgtgccaggtcttgctgatctcgaagacccggtcgttggagttcttggcgaagttgacccggatcagtttgggtgccccggtggccgcccgggcgtcggcgagggcgcgcgcgatctccaggtcacgggggaggatgccgaagttggcgtcgcagacgaagaggtcctcgacgtcgtgttcggcgaaccactcgatctcctcctggaggcgttcggcgtcgaagagccgcagcgcgctcatggtggccgagccccagtcgcagaaggcacacgagtaggggcagccgcggttggtctcccacagcgcgtagaaccgcaggtcgcgttggcggcaggtggccaccgcgccgtccatcacgcccagcaggtaggggctgggggtctcgatgcggcgcggcagccgctcggccgggcggccgggcaccgcctcggtgccgtggcgtaccgagacgcccggcaccctggtgtagtcggggtggtcggccagccgctcgatgagcagttcccggaaggccgtctcgccctcgccgtgcacgagcacgtcgacgtaggggtggcgggcgaagaagtcaccgggccggtcggggacgtgcgggccgccggcgaccaccagcatccccgggtggcgttccttgaccagccgggccaccttcatctggcggcggaagttccacacgtagcaggagagcgccagcacgtccgggtcggtgatcccggcggccatctcctggaccccgtcgaccaggaacaccggttcctggaagtcgtatccggccgcgacgcgcggatcctgttccgcgtaggtgcgcagcagaccggccaccaggggcaggaacggcaccgcgttgaggccgatgaagtacaccttgcgaccgctgcgcgcggcggggacggtcatgctctcacggctcctcgggttcggggctgttgccggctgcgggcaccgggcggtgtcatccggtgccggcgaccgtctcgggctcgcgcgtcggcgaccagctctcgcgcgggtgcgaccagtcggcgccgcgtcgcagttcccgcagcatctggtcgaaggcgtcgtcgtcggagtagtagaagaccgacgggtcgctctccccggtcaccccgggcgcggtacgggcgctggcgccggccagcaggtcggccagttcgccgtagaaccgctccagcggcagccgggtgcggaacagcgagtggtagcagtcgaagagttcggggttgtcgctgatcagcccgagcttggcctcgtcgtacaggtcggtgcccggcagcggggtgagcaccgagaagctgggcatgtccacctggagttcgtcgaccgtgcggccgagccgggcgaagtccgcctcggaccagtcgggttgcacgatgaagttggcgcggaccttgaccccgttgccgtggaagaggtccagcgcgcgcctggtctggccggggtcggtgcccttgcggtagccggtcagttcctcgtcggtcatcgactcggcacccaccagcaccgagtccagcccgatctcggcccagcgggcgatcacgtcggggcggcgcagcgcggtgtcggtgcgtacgtacatgtggaacttcttgcggaagcccgccttgtcgatggcgtcggccagttccagcatgcggaccggctggatgaacgcctcgtcgtcgacgacgtagaggttgtcgccgtcgatgtcggcgatctcggcgagcacccggtcgatgtccttgaccaggtagcggcggtcggtcatccgccacagcgagcagaacttgcaggtgtacgggcagccggcggtgaagcggatgagcgccaccggccggtagatggagtggaagtagcgtccgcggtggtgggccaggcgggtgcggtccggcatcggcaggtcgtcgagggcggacggcttggggcgccggtcgcgttcgagcccggtgcgggcgtcgtagggggcgaggccgcgtaccggggcgggggtgcggccggcctccagttcggcggcgaggtgggccagtggtccgccgccctcgccggccacgatccagtcggcggcgaacccggtcagccactcggcgctgacggtcgggtggtggccgcccagcacgatcggggtgcccggccacagccgccgggcctggtgggtcacgtcccgggagacccggacgctggtggtgtaggggatggcgacgccgatcaggtcgggcgcctcgtgggtggcggcgtactcctccagggaggtgtccacccgcaggtcgtggatggtgacgtcgtgggcgccgcgcagggcgcccgccaggtattccagcggcagcggctcacaacagctgagccggtcgctgaggagggcccgctcgttacgaggccacaccagcaagaccctcatcggatccctccaactcgtcggtaccggcagcggtgtcgcggaagacctcggtggccatctcgtacagccgccttgcctgggtgtgccggtcgtcgctgcccagggcggcggcgaacatgcggctcatggcggtgccctcgtggatgccgcgcttggggcgccaggcgacctcgtccggcaggatgccggtgaccgcgctgcgcagcacccacttcgcggtgtcggggcgcaccttgagccggccgcggatgccgagcgcggtggacatcacctcgcgggtccagtacgggtagcgcaccaccacgtcgcgggcgtcggcgtaggcggggttgaactcgttgctgacggcggtggcggcggtctgggcggcgaccgagcgttcgatccgggtctcggtgccggtgccgccgaggccggcgaagatcaggtcggcgccgtaaccggagagcatcacctggcgggcgccggtggcgatgtggcccagcaggaagcaggcgggggcggcgatctggagggtgagcaggtcccaggtctccagcgagcggatcatctccggcagcagcgcggtcagttgctccggttcgaagaccagctcgtggtgtttgctgccgagccgttcggcgaaccaggcggcctgcgcgaactcgtcgccgaacggggtgcccacggtgtacgtggacagcgaggtcacctcgcgggcggcgagcgcggccaccgaggaggagtccacgccgccgctgagcatcaccccggcctcggtcagcccggtggcgaggcggccgaccgcgtgggtcagcgcgtcgcggacggcggtgacggcgcgttcggggtcggtctcggtgggccgggcgggcagccggtggtagcggcgggtgagcaggggctcggtgtcgaacccggccaccgcgagggtgaccgcggtgcccgcggggacggcggtgaccccggcgaaccggccgccgggcagcaccgcgcggtcgccctcgcccagcaccggagcggtgcccagcagcggcagcagggcgccggccgaggtggcgaccagcagggtgtcgtcggcgccgcgggcgtagtgcagcgggcaggtggcgccgttgtcgctccacagcaccgcgcggtcgtcgcgtaccagcaccgcgcagaagcgggagtaggcgcggaccgtccaggcgatcagttcgtccagcggggcgtggatcggcgcctggccgcccggggtgagcgggtcgccgtggaccacgaggacgtcggcgccgtcccgggtcacctcggtggcgagcccggtgccggcgccgaggtgttgcgggacggcaccctccgggcggcagcgcagcaggaatccggccatgtcagtagaccccctcgatccggcgtccctcggtgaattcggggctgtacgggtcgcccagccagggccacgggtagcgttccgcggccgctccgtagggctggacgcggcgggcccggtcgcgttcgaggaagccgggcagcagggcgtcgtcggtgagccgccagacgcggacccggccctcctcgccgaacggcacctcgttgccgtcgtcgtcgaccgcgtccagggcgatgtgcggctggcagggcacgtagaccacgtggtggtcgtcctccggctcgaacggcttctgccagctgatgccggtggtggaggtgccgtacatgccgaccacggggaccccggggaagaagtcctcgcgcagttggcggtgggattccggctccatggtggtgccggcgtggacgatggcgcgaagccccggcagcgcttcggtgccgaggcgttccggcagcatctccagcagccgtgaggtgcagaagagcaccccgatccgctgggcgcgggcgacttcggcgacctggtcccagatgtgctggacgtagcggtcataggcggccgtcatgccttcctcgccgaagatcttcacgatccgcgggtcgaggtcgacggagaagcacatcccgccccggcgttccgcgaaggcgaccaccagccgtccggtggtgtgcggtcccatcgggccgacgaagagccagtcgacgccgcgcggcacgccgtgcaggtcgaggaactcgtcggagaactccaggatgtcggcccagtaacgggcgccccaggtgccgtgtttggcgggtccggtggtgccgccggtctgcgcggcccacagcggtccgccctcggtgaggacgcggcgcggcacgaagttcgccagtggcgtgcgccgggacgcctcctcgaagtgtgcctggtccggcccgtcgcgcaagccgatcagttgctgggcgtccagcatgccgtgcaccttgcgcagtgcgtcggtgccgtcgcgccggtcgcgctccagccagtacggggtgccgtcctgcgggtcgaagtggagtcgtacggcccgtcgcacgtcctcgtcggtgatcggacggagcttggacggacttggcatcagcagtctccctcgtacgtcgctgtcctggcttccgctcacccggccgcgcccgggatgaccgccttcttccggaacaggaagtcggcgaggtagccctcgtgcgggtcacacgggtggtagccgcggtcgtactcctcggcgccgaagaccttgtccaccccgggctcctcggccagcgcgccgagcagtccggccgcgtcgccgaggaccgagacgatcagcgaaccggcgcaggcggccaccgccttggagcgcggcacgtgggcaacggccgtgaacgggaacggcagttcggtgccgaagagcggtgaccccgggtcgacaaggagcacggtggggcgcaggaaggcggcgccgtccacctcgacccgcagcggcaccccggtgacggcctcggtgacgtcgacggcaccggccgcgatctcccgttcgatgcgggtggcgagggcgtcgcgccgcgcccggtccgggaacgccggtacggtggccgccgggtcgtccagcggcagcaccggccgggtggcgaactcctcggcgaggcggtcggcggcctggcgggcgtccccgagggcgatcaccgagctgacgttggtgcacagccgtccgcagccggcgaaggcggcccgggccagccggggccacgcgtcgtcggcggcgccggcgcccaccaccgcccggctgcggccgaagtggtaggtcttcgccttgcccggcggcaggtcggcggggacgtcctcgccgggccagagcacctggtcggcgaggcggcgcagcgccggggcgtcgccgtgcaccaccgagacggcgtgctcgggcagcccggcctcgtacagcgcctcggtgagcagcaccggggtgaacgggtcgtgccgggaggtggacagcagcaccgggcggcgggcggcgagcgcctggagccattcgatgttgatggtggggaagttgtcggcgaccttgaccagcaccgagcggccggccgggcgccagctccagcgggggtggtggccgccggtgcggtaggccgcgacggtgccgtccggtgactgggcggcgaggatctcgtccatccgggccaggtcggcggcgacggtctcgatgccgcgggcggcgcgccggggaggcagcccgctcgccgcggagagggcggtcagccacggctgcgcggtgccgtcggcgacgcgtgcggtgagcgtgtccgcggcgcggcgcagcagcgcgaaccaggcgtcgtcgtcggtggcgcgcagccgccgggcgaccgcggcggcctcctgggccatctggaactggacgagccggccggccgccccgacccgggccaccgtctcgccggtggtgccggtgagcgcggccggcctgctggacatcaccggccggcgcccggcgtaggccgggacgtcgaggacggcgccggcccgctgcgggccggcgtgtccggtggtgacggtcgtcactggacctccggtaccagtgcggccggttcgttcgacaggttgctcgcgccgtcgccgtcgagcaggccgaggcgggtctgcacggtctggaggttggcgaggaacgcgatccgcttgtactggccctggctgcggctgggggtcatgtcgtcgtagagcttccacagctcgtcgatggaggcccccatgctggaggtgaccgggatgtggccggtgtagcagtcggtgtcgctggtctcgacggtgaagtcgacccgcttggccatgtccttctccacctcctggtggaaggggacgaagaagcagggcgagacgaggtcgtgcttgttcttgccgagccgggcctggagccaggtgatggtggtgcgcatcgcctcgtcggtctcgccggggatgcccaggatcatggtggcctgcacgccgatgccgttgttggtcaggcagctgatggcgcgttcggtgacgtccaccttgaccgggcggccgagccgcttgagcaggttccagctcagtgtctccacgcccaccagcagccggaagcagccggattcgcgcagcatgcgggccatgtcgtcgttgatcaggtccgggcgggccttggcctcccactcgaacttgccctggagtccggacttggcgatgccgtcgcacagctcctgcatgcgcgggacgtcctcgacgaagtcgtcgtcctcgatgcgtatccggtcgaagccgtacacctcgaccagttcgcggatctcgtcgaccacccgggccgcgctgtgccggcgctggccgacgccccactggtgcttggagtagcagtacgcgcaggcgtaggagcagccgcggctggtgaccaggttgccggagcggccccgggagatccgccggtacaggtcgaggtcggccaggtcgcgggcgggcttggggagtttgtcgaggttgaccacctgcggggcggcgggggtctccaccacttcgccctcgtcgtcgcggaaggtgatgccgcggaccttggcgaagttcttccggccgttcttgccgaattcgtggacgagttgggagaaggagtactcgccctcgccgcgcaccaccacgtcggcggcgttctcctcgatgagcttgcgggcgatggggctggcgccgacgccgccgagcacggtgagcgcgttggggtggcggtggcgtacctcggaggcgagcttgatgccgttcttcaggctggaggtgagcacctggatgccgacgatgtccggccggtagccgcgcaggaactcgctgggggtggacagcgggcgctggaggacgaagagctggtcgtcgatgaagtgcacgtcggtgccggcggtgtcgtggaaggtcccttcccggagcaccgaggcgagcgacagcaggcccagttgcggagcggagatcgactctcccgagtacggtgaccggcgggcgttcacgaggagaacttgcatgtcaggtctgcctcctgcggttggagagccccgggcgtggcggggccgaccgccggcagccccgcgcacacggggattccgtggcgtgaagaagcgctcctgcccggtcggcggcttggcgccggggcagcaggacgggttcgaagccggcggcggttcccgggacttgcgccgtcggtcagcggcagttgggaagaagtcttcaccagatgaacatgggccgtaatgcccggtccaatgcctttaccgggtcgcgccaatgccgtgaggacatgggatcggtctggaacgggcggccatgcccgcgggccatcaccgcgatgcgccggggacactccctgcggccgggttggtgtggcagcatcgctgaggccagatcacgtgcgctcgtcggatggtgatgggaaggtgcggatgaccgcctcacatgacacggcagacgccacggaccggaccgtcggttccacgtccggcgaacgagcggcgttcgcccgtgacggagtggtgcgttggggccgcctgctgacgcctgatcagatcgacgcgctgcggtcctcggtggagcgtgccttcttccgcgacggccaccccgccgacggggtacgcgatctgtccgaacgccagggccgcccgctcgacctggccctgctgcacaagatcaacctgtggcggaccgacgaggcgtgcgcggcccaggtggccagggccgacctggcggaccgggccgaggcgctgctcggcgggccggtacggctctaccgcgaccacgtcttctacaagccgccgggcaagggcgaccgcagccgcatggtgctccaccaggacaaccgctactggcacctggacccgccggaggccatcacggtgtggatggcgctggacgacgccaccgtggagaacgggtgcgtccactacgtcctcggcagccaccgccacggccgggtggagcacgtgcgtcccgaggagggcgcggtgatgatcgaggcccgtaccgagcaggagccggtggcctacccggcccccgccggggacgccctggtgcacagcgtcaacacgctgcacggctcggggcccaacctcagcgacggaccgcgccgcgcctacgtggtggtctacgtgcgcgacggggtgacgatgcgcggcgagccgatgacctcgttcccgctcgtcggcgacctggtccgcggcgggtgagggcgcgccccgtcaccgcgacaccgagccgcccgcaccggaccgccctccggtgcgggctttcgccttggcgcgtacggaaattgatggcccgtcagcgaccgaaaccgccgccgtaccggcgcagcgcgtccggcagctcctcgatccgggaggtgtcccacggccgcccccagccggccagccgcatcagccagtcgacgatgctcccggtgaacccccacacgaacagctcccgcaccaggaacccggggctgatgtaaccgtcctggtagcgcacccggaaccggttggcggggtcggcgaactcggcgagcggcacccggtgcaccgagaccacctcgccgcggtcaccgcgcggttcggcctcggcgccccaccagccgagcaccggggtgacccggaagtcggtccaggcgatgcgcagcgggcgcagggtgccggccaccgcgacgcctgccgggtcgagcccggtctcctcggcggattcgcgcagcgcggtgtggaccacgtcgcggtcgcccgcactcaccgatccgcccgggaagcacacctcgtcggggtgggagcgcagggtgcgggagcgtcgtacgaggagcaggtcggtcccggcgtcgccggtgccgaagagcaccagcacggcggaggggcggccccgggcggggtcgtcgaggaggtagcgcaccagctcgggagggctgtcgtcggccgcttgccgggccaggccggtcagccaggtcggaacatcagttcggtgtcgtgggtacacgcgatcgaggtccaccgggtcagcgtaggcaccggggccgtccgccatcaccctgatcgccgaaaaagacactcccccgtggactcccggtgcgcggggtccccggggccctgccatgatgggcggcgcagccaccgagagtgaggtcgcgtcagccatgggtagtcaccgtctcctccgcgcccgcgccgggaccgccggggccggccccagcagacgcgggctgatcggcacctcgctggcggggatctccgggctcgccgccgggttcggctccctcggggcgcgtccggccgccgccgcccggcccggtccggtcggcggcacgacccggctgcggtggttcggcaccaacgcctgggagatcggcttcggcgggcgcaccgtgctggtcgacccgtggctgacccgcttcaccgcgcagcggccggacggacgggtggacccggacaccccgctcaccgtcgaccattccgccgtcgaccgccacctgcgcgccgccgatctgatcctgctcacccacggccactacgaccacatcggcgatctgccgtacgtgatgcggaagttcccgtccgccccggtggtggccaccgagacccacgcccatctgctcaccgccatgggcgcccccaccgaccgggtcatctgggcccgtggcggcgaatacctggacttcggcgacttcgcggtacgcgtcctgcccagcctgcacagcatgggccccgaccaccgctacttcgcccccggcacgctcaccgcgccgccgcgccgcgcccgtaccgtcggcgacctgctggagggcggcacgctcgcgtaccacatcacctcggacagcggggcgagcgtcgtcaacatcggcaccgccaacgtcatcgagcgggagctgaccgggctgcggccccacgtggccatcgtcgcggtgccgccgtgcggggccacccaccgctatctggagcgggtgctcaccgcgctcggccatccgccgcacgtgatccccacccaccacgaccagctcgacaccccgctgttccggccggccagcgtggaccccgggcagatgcgttccttccgcgaccaggtacgcgcgctcggcccggggtgcgcggtgaccgaaccgcagtactgcgacgcgttcacgctctgatcgcggatcccgtgccacgggggtgttcccgggccgcacggtgtgctggagtggaccccgaggccggatcaagggcccgcgggctgaagggagttcaccgggtgagcgcgaacagcgggggcatcggcggcgtgccgggtccgcacaacgggctgaccgacgtacccggggtgcgggtcggccacgccgggcgtaccggggacggctggctcaccggggtcaccgtggtgctcgctccgcccggcggtgcggtggccgccgtggacgtccgcggcggcggccccggcacccgggagaccgacgccctcgatccgcgcaacctcgtgcagaccatcgacgccgtggtgctcaccggcggcagcgccttcgggctggacgcggcgggcggggtggcggcctggctggaggaacagggccgggggttcccggtcggcgccgacccgtcccaggtggttccggtggtgcccgcggcggccctgttcgacctgggccggggcggtacgtggcgggcgcggccggacgccgcgctgggccgggccgccgtcgaggccgccgccgcccgcccggagggtgacccggtggagcagggcggggtgggcgcgggcaccggcgcggtggtcggcgggctcaagggcggcatcggcacggccagcgtggtgctggactccggcgccacggtggccgcgctcgccgccgtcaacgcggccggttccgcggtggatccggccaccggcgtgctctacggcgcccgtaccggactgcccggggagttcgccggatacggggtgccggacgcgatcggcgccgacacgcacgcccgggcgcgggcacggctggccgaggccgccgaggagaccgcccggcgccgggccggcggcgccgccaccctcaacaccacgctcgccgtggtcgccaccgacgccacgctcacccgcgcccaggcccagaagctggccggcaccgcgcacgacgggctggcccgggcggtgcgcccggtgcacctgctctccgacggcgacaccgtcttcgcgctctccaccggccgccggccgctcctgcccgaccggccggacgccacggcggcccgcgccttcggggtccacctggaggccggcgcgctcaacgaggtgctggcggccggcgccgacgtcctcacccgcgccgtggtccacgccgtcctggccgccaccggggtggacacccccggcggggtccacccgtcctaccgggagctgtacgcgcgtccctgagccgtgcccgaagccctgccgccgtactcgaagccgtgaccagccgcttcgttgaccggccaactgccgctgcctacactgcacttgacgaatccttgacacaggcttgaccggcctgtgccggcggtcccgccgcgttcgcgggtttggggggatccactgatggacgcagacgactgttgggcgcgggcgggcaccgtgcggatccgcctgctcggcccggtggagctggcctgcggcacgcggccggtgccggtgaccgggcggcgccagttgagggtggtggccgcgctcgcgctggaggccggacgggtgctctccaccgcggggctgatcgcctcgttgtgggcggacgagccgccgcgcaccgccgcccggcagctccagaccagcgtgtggatgatccgccgggcgctcgcctcggtgggcgcgccgcagtgcgtcgtccgctccaccccggccggctacctgctcgacccggcccactacgaactcgacagcgaccggttccggcacgcggtgctgaccgcccgggagttgcagcgggacgggcggctggcccaggcccgggcccgggtcgacgaggggctggcgctgtggcgcggccccgccctcggcgcggcggcgggcgccggactccagccccgggcccgccggctggaggaggaacgggtcttcgccctggagcagcgcgccgggctcgacctcgcgctcggccgccacgagacggccatcggcgaactcctcgacctcatcgcccagcatccgctgcgcgaggcggcctacgccgacctgatgctcgccctgtaccgttccggccgccagtccgacgcgctcgccgtctaccgcagggcgcagcgggtgctcgccgacgagctggccgtccgccccggcccccgcctcgccggcctggagcgggccatcctgcggcaggacgagtcgctgctggccggcgcggcggtgccctgaccggcccgtcccggcggggggcgggggtcaggggcggacgtaggggcgcgtcatgacctccatgttgtgtccgtccgggtcggcgaagtacgcgccccggccgccgaacagcccgttgacgcggcccggttcggtgtgcgcggggtcggcgtagtacgtcacgccgacggtctccagccggtcgatcacggcgtcgaaccggtcctcggggacgatgaaggcgtagtgctgcgactggatcggctcgtcgcgcttctcgtagtagtcgagcgtcaccccgttgccgaggtcgacggggaggaacggcccgaacggcttgccgacctccaggccgaggatcgcggcgatgaactccgccgacaggtagcggtcggaggcgtagacggcggtgtggtcgaggcggacggtcgcggtgggcgacgcggagtcgtgcggtgtcatcaggtgtctctccagtcttcggaaactgcgggagcgaggcgccacgcgtgggcgccggaaacgaagacacggaagacgggcgggcctcgtccccaccccggcgtcaagccgaggccgggaccctcactcgtgcgtggcccaaggccgacccggcagtcacgtccgtgatcataacggcggtccggacgcccggacaacgccgttaccgggcgcggggcggtcagcccccggacgcctgcggggggcggcgcagcgggccccagtcgccacgctgccgggcgacctcgtcgcgggcctcgtggatcacctcggggtcgatccaggcgatcgggcgtacgtcggtgagcagcggttcgttgtcggggccgcgtcccacctcccggccgcgcagcacccaggggcggaccccgggaccgatgtcgtggcgcaggtggcagtagtcgtacaggcggcgggccacccacagccggagcgggcggccggcccaccagtcgtccacgtcgagcgggttcgccgacaggccgggcagcgagatgccggtgaggctgtcggcgctcccggtctcccgcaggtcgtagcgggggccgcgggaccagcgcacgtacagcccgcggctgccgtcggcgagccgcgcgaggtcgtcgaggctgctgagcacgggcagcccggccgaagaggtcacaggcgacctccggcgggcgggtacggccgaccggacatgtctgccgcgtaccccggaccacgccggtgacacccggcgcacccgtcccgggcggtacgcccgccggggcggtacgggtggccgccggaacgcggggtaggcgcggaccatgaacactctcgcatggtggttacggctgacggtcctggcggtggcgggcggcgcggtcgccctgctggtacagcactggacggacgggcgcccgtggccggaggcgggcgtccccgctgcgctgctggtcgtggtggcgctgctgatgggggtggtctcccggcggtcgcggcgcgatctgtggtgacaccccggggcggcggctacgcgccgggcgaacaccggcggcgtcccgcgctccgcacggcacagtcggatcatgacgaaccgaacagccgtactctccgacatccacggggtgcttcccgcactggaagccgtgctcgccgagcccgaggtgcgggccgccgaccgcgtcgtcctcaccggtgacatcgcctgcggcccgcaaccggccgaggtgctcgacctgttgaccgcgctgggcgaccgggtgacctgggtcgccggcaacgccgaccgcgaactggtggagttccggcgcggggtacgcgagacgatccccgaccccatcggcccgtgggccgcgcggcagctccgccccgaccacctggagctgctggcctccctgccgctttcggtgcggctgccggtggccgggctgggcacggtgctcttctgccacgccacaccacgggacgacgaggaggtggtggtggtcgactcgcgtccggaccgctggcgtgaggtcttcgacggcctcggccccgacgtggacgccgtgatctgcggccacacccacatgcccttcgtccgcctcgcccacggccgcctggtggtcaaccccggcagcgtcggcatgccctacggccgctccggcgcccactgggccctcctcggccccggcgtcgacctgcgccgcaccccctacgacaccgacgccgccatcgcccgcctcacccgcgactgcggctaccccgccatcgccgagtgggccgactactacgtgcgcgcccgggcgacggacacggaggcgctggcggcgttcggcccgagggacggacgcggggcggaagggtagtccaccgggggcgcggggcggtcggggtcggggccgccgggtgtgcgggacgatcggcgctggggcgcggggcgttgtcggtggccggtgggatgctcgccgtcatgacgccctggaccctcaccgccatcgaacgtgccctgcgcgccagttgggccgcggacacctgctcccccgacgatctggcccgcgccccgtggtccccgggcaacccggcgtgggggcactgcgacatcaccgcgctgctcgtcaacgatctgctcggcggtgacctggtgtgcggcgaggtacggctcggtacatcagttccggctcgggtagagcatcgtcgcgccgcgcctggtacgccgtcccccggggccgacccggcatcgctgggcggaatacctgctgctgcgcgagcgggtggtcggcaggctcggcgtgctgcccgaccctgcggggaccaactccacacagtgaacaactcgttcacatcaacccgttgacaatgcccgcaaccgaacacatgattgactcatgcctgagtccatcgaagatccgttcaccccgcccagccccgagcaggcgcgcgccgagcgcgtggccacctcgctgttccggatagccgagcggcacgccgcgacggaggagcggcggcggcgtcagacgcatccgtacgtgctcgctccgcacgaggcggtgcggctggtggccttcctgctgagcggggccgcgcagcacgaggaggacgagccggaggtggaccgcgcggatctgctggccgcgctcacgctgctgccggcggcccgcgccgacctggacgagatcgaggccggcctgctcaagatggcccggggccggggcctcacctggccggagatagccttcggcctcggcctcggcaccccgcaggccgcgcgccagcgctacgaacgcctctccggccgcatatccgccgaccccgagtgaccaccggccgcccccggcgccaccgggaccgtccagaccgcctttaaggaagatttacggtcccgttggcaccgaaagatccgtcttcgcccgggcgtggttgtggaacgacgcgctgattggggcgagttggccggttcccggccggttatgcgcggccttgacgccctggtggcgcggtggttccctcagtggccgagtgacgcaacgtcgcgcttgcggccccgggtgcccacgacggcacccgcgccgcagccgctggaagccgcaacgtccgcaatccgcactccacccggtccacgcagcaagcaaggcgcagcagcaccgcccaccacgggcaacgcaccggtgacgcgcgggggacggcgcgtacccgcgacggcacggcatgccgcggagccctgcccgcacctccccgccgacgacttcacccacgcccgcgcgcgtcaccgcgcgggcgcacccggcgctgcccccggcccacccaggaggacgccatgttccaccgcatcagccggcccggccgaggattacgcgacggggccggaacggtcacacgcggccgaagacaccgcccggtacgtgggccgggcgagcggtcgcggtgttcgcggcccgcctcggccctggccgtggcgctgacgctggcgctcaccgcgggggtggtctcccccgccatcgccgcgacgacgcccagagccgccccgtccgccgctcccccgccgcccgcgccgaccaccgccgccggcgacgtgctggtcaacggctggggcgacggcgccggttaccacctggacgtcgccaccggcagcggcggctacgactggcgtgagctggccgtgctgcgcccggcgggcatcgacgactcgtcctggacgggctaccagtgcgtctccggcgacgggcggtacgcggcggtggcgatcctgcccgcgtccgcggtgaacctggccgtcgcccgcgaccacggtgccttcgcctactccgtcgacctgcgctccggcgcggtcaagccggtggcggcgggcgtggcgctcaagtaccacaccccggggtgcggcaccggcgacaccgccgagttcaccatcgacccgggcaacgaccagcgggccacccaggtgctctcggccgatctgccctccggacgcctcacccacgtcaccaccgtgcccggccaggtgacctccgtggtccccgccggtgacgggccggtcggggtgctcggcggcgaactggtgcggataccggaggacggcggccggacggcacgcccggtcgcgctggcgtcggtcggcgggctcgcctacgacctgcgtccggcggccggcggcggcgtggacttcgcggtgcaacgggccgcgggccgcagctcgttgatcgtccgggagcgctccggacacctcaccacgctgggcgagggcacccacaccggtctgcggctgatgcagggcagggcgggccacgccctggcgctggaggccacccgcctggtcccgggcagcggggtgcgcgccgtcgccacccgtggtctgccgggtgccgccaccggtgtctcgctggacggcggcgcggtgctgggcctcggcgccaggaccgccgcgtcggcgccactggtgctggccacccgcaccggcaaggtgctcaagcgctccgccgcgccctcggcccggcggccggacaccgtgctgccggtgccgccgcccggcgcgttcggcggtgccgcgggcgggctcaccccgtcgtcggcgggcgtcagggggacggtctccccgctgaccgcgacgcccaagtgcgcggtgggccgcaacgaggagaaccggcaggtgatgcagcccggcacggcccaggtgtcgtgggcggtgcagatggccgagcaggggctgctgaccggctcggcgtaccagcgccccgccaacttcgccaacctggggctcgtcgcctactcgcccaacggcgacttcggcaaggtggcgctccaccacccgtccggtgacagctgggactcggtgccgcgctcggtgtaccaggccatcgtggcccaggagagcaactacagccaggcgtcctggcactcgctgccgggcatcccgggcaacccgctgatcgccgactactacggcgccggcggatcc

SEQ ID NO: 2 - protein, 250 aa, Streptomyces cattleya
MIPRSFPALGDRGGGARHRPADRRPDRAAPPGGPSAGHPLPAFLRARRLRSGLSQEGLAWRLDVSTRTVSNWERGQHGVDPRRVADLAAALALTREEERELAGLAGSRTPAARPAPAPETPHPGDPAGFAREWHRGFRDVGMPAYLRDPAWRLLACNAAYTRLFSGVARVPEAIPRENLARFICLHPDAPRLLADWYEGWLVPLLCEISDDLRAAPPRSPLYALLKAVLARPEIARAWRTEVPARAAARI

SEQ ID NO: 3 - protein, 296 aa, Streptomyces cattleya
MTDAQPSGPVVEHGRGDLTAEERRQKLSFLREKRDSVDRSSFEARYPVEVPRRLPGRGRHAPGLTLVEMGRLLGLRDGSWYADLERGQVDNAPVDRLFKVAELLDLTPRQYEFLCVYACRPKPGYPAAGLPGGYREVLDLYQGPAYFQDAGWNLLRPKMYNAAAEALFNGIPDDFNFIRWVLLERSVRRPPEGGRRHAWLPDFWQVWAPRALPVVRAAYVARPDNATLARLHADLAADPEIGPIYQGRLPPYPHTDGDLRPFVHGSTGERGMVRINVLTPRGSDDTLMLLNWHPGA

SEQ ID NO: 4 - protein, 259 aa, Streptomyces cattleya
MDLRMSGKTAVVTGASRGIGLAVVRTLTGEGVRVVGAARTVTAELKDAGAVPVAVDLSTPEGCAELVQRALAELGGIDLLVNNAGGGDHFTAAGFLTADDEIWERSWALNFFAPVRLIRAALPSLIERRGAIVNVSSIGARNATGPIDYATAKAALNTLGKALAAEFGPRGVRVNTVSPGPTRTPVWEDPDGYGAQQAALGGQELADYVARVPARSGQLTGRLVEPGEVADLIAFLGSDLAASVHGADYVIDGGALKTV

SEQ ID NO: 5 - protein, 360 aa, Streptomyces cattleya
MARLTRAETQERNRDKVLDAAREEFAERGYRDARIDAIAERAGLTRGAVYSNFPGKRALYFAVLAALAERVPEGPPPRPVRSGGDALAAFARAWVSRLPPAGDDGPGPARLAMDLMPEVITDAATRRPFAQLLKLDAILLGLALERLEPAGRPARRMVRVAEAVLTALHGTAQLAAAAPGFLDPLDAVRVCERLAGLPLDDDPPPAPGLPPVHHVDEPWSPPPATDAVTGDPLPADGEPDQVVTVLGLHRLEAAEDAVRAAPPDTPVTAVLVTADPGELGALARLSVAELCGCLRQAFPASARPPLRVVHDPSGAFAAAAGVPAVDDRTETAIRLASARVTARAQGPGAARAVAATPRRG

SEQ ID NO: 6 - protein, 212 aa, Streptomyces cattleya
MEPMLTTALAFLGACVLIAAAPGPSTMLIIRQSLHSRRAGFLTVLGNETGVLTWGVVAALGLTALLAASRTAYDVMRIGGAVVLVWYGVQTLRAARRGEARPSAADDEAAVVPRSGWKIYRSGLLLNLANPKAAVFAMSFLPQFVPAGAPKLPVITALAAFQALFEVGYYGMYVWFVGRMKRVISRAGVRRRLEQVSGGVLVLLGIRMAVES

SEQ ID NO: 7 - protein, 363 aa, Streptomyces cattleya
VTLPTETWAVRIHRHGGPEVLVHERLPLPPLGPADVLVAVDTASVSGWDVKYRRGLPPGARLPGRERYRLPLQLGREAAGTVLATGPEAAGRFRPGDRVVAVVHPENPRAPETVRGLGNLSTGIALPGHQAPGGYARYLICHQDMWLPLPSGVDLEQAAVTLWPYATCHRVLRDRLRVALGETLLVCGATGAMGLAALRLARLTGVRVIAMTRYRAKERALRTAGADEVVVAGDPRQACEAVRALTGGEGVDHAVDFTGSAALLRLAVDSLRLGGRLCPAATQRPPGPLPVTTGDLTRLEITVYGIRGARHRDALRVLALLGDGSLPATPIAARFPLSRAGAAHEFLEHNTTAVGRVVLKPGW

SEQ ID NO: 8 - protein, 294 aa, Streptomyces cattleya
MGAAAGERRTLSRKRGTTVDVPVKQESRQDRFEDQDEDRFAAQESIECSRLGDGIALAEFSGAHEQNPFSRARMRELTALMRELDADEKVRCVVLYGGAGRSFGVGGDFHEVSEFTGGDEVNAWIDDITDLYTTVAAISKPVIAAIDGYAIGVGLQISLCCDYRLGSEQARLVMPEFRVGIACNFGGFMLEAAAGRTVMQRMLLTCDEWPAERALADGLLHETVASPRLLDRALELARTISGYTAEAVQSTRPRVNAPFVAGLERIRREAKESHRRAFAAGEAQVRMRRVIGRS

SEQ ID NO: 9 - protein, 327 aa, Streptomyces cattleya
VSPRRLVPIRPADHLPALVRLAADRDRAALGRVEVGATWIAGRLAAPGLDPEHDTALLTGSDGQVAGAVWLSRSSGRSAWDAELLLAPHATPDDAASLLDFAHRRCRTIAAGRPEGEVCLSCFVSERETALRIALSAYGFAAPRPYLRMSVPLGGATAGEPPVPGAVVRVPEGEAGLRAFHAVKNRAFSAEEAGMEPDGFEEWLRWTGADPGADPSQRALLELDGEPVGFANVTDRMAQTRGAAYLRQIGVLPRMRGRRLGAFLLRSVMAAARARGRGEMVLTVDTGNRAGLALYRATGWREESRFYDYAYRVTATPAPAGEVSSAR

SEQ ID NO: 10 - protein, 263 aa, Streptomyces cattleya
MPVTQSIQALSDELRRDGVVALDDLVPESVLEVMRRGCAELIGRTERMRISTEEWQLEAEGEGGGWAARAAGKECIPGKVRIIGRAHEHSADLAAVPDLVGLNEKLIEPLHGLPGDFYDCFLWAKPSRVGSQKPWHQDAIFLADEWHEKYVDVYTIWIAVDDAREDNGCLRFLPGSHRERKLYEPDGIDPGDIFASPREPSLDIARIWPGLTPVTLPRKAGSALLFNGYVAHTSAPNTTEDDRRAVSYCYSLPRHPAGPESGR

SEQ ID NO: 11 - protein, 224 aa, Streptomyces cattleya
MVCAASSPAGALLVDWGGVLTQPFYAGIAEWAARDGVDADAFHALLARHLGPGAPGGAAASVFHRVERGEVPVAELEVTLAESLRRPDGTGPPAEGLIQRMFQPFTMAGAMVELVRRVRASGAAVALLSNSWGHTYDRTGWDGLFDEVVISCEVGMRKPEPEIYRYTARRLGVAPRRCVFLDDLGRNVRAAAAVGMTAVQHTSVEESSRELARFFDVSPLPAGR

SEQ ID NO: 12 - protein, 476 aa, Streptomyces cattleya
MFDLRRSDVALLQAVADHGSLHAAQRQGWVTQSSASRRLTRLERRLRTVLVSRGSTGARLTDEGHALLHAGQRLLGAIDFAMANAADTDEPGPRLRVLQMAVSTEYTCEAGEEPAVGFPDVVFDLVPAARQDVWSRFDRYAVDAACGWARSTPALRRYGADVRLVVEEPQWVAVPRGHPLAGRGALGLEDLAEAEWVAIVGESTHEDLARAFARHGLTPRVGCTVHSRSAAADLVARGYGFALVSPLCAAPVEDAGYALRPLRQQVVRRLFLATDPLLLPEALARDLCTGLQRRYVQHAAERNPGYLRSTTFPLPAADLSAPPPGRSAATEPDGPRDPLPFLVCERHWTAEGPSSLVEAEHLYLLRVIERTGSLNRAASDLLITQPALTRRIHRLEQVCGRTLVHSAPRGTCLSPMARRLLHCTEAAEDRLDTLVAALRRAAASTTPPLPRQRPAGSGETSKNRASSLLLSSTEVC

SEQ ID NO: 13 - protein, 483 aa, Streptomyces cattleya
VSAPAHGSPARGASTVALAVLASACGLFALMQLAVTMLLTQLRHGFGVGAADTGWVVSGHLLVACVATPVVGRLGDLYGRRRVLLAVLAVFAAGGAVAASADTFGLLLAARLVMGVAGGLFPLAVGLVREGFPAGALAGPFGVLSASFGVGGGAGIMLAGVVEGDPDGYRWVFATGAALAALLLLIGAAVLPETPRRAGQRLDAPGLTLLAVSSALVLLALGRYGRDGIGSVTGSTLLAGAVVSGAALLAWERRTTTPMMDLRLMSRRPIWTLNAVSLLTGLVMFVTGTFPPAIAEAPRSAGGLGVAGLGVIVVMMPMEIGTLGGGLLSGRLGRVLPPRAVMVTGTVLFAASAVVFAFLPVSMASLALGTLLNGAATGVLGGAMTELITLASPGGTTGVVVGTNSLLRSLGGSFGVQAGSMVLAAGTTGDHPSPAAYTLVFAGTAVLGALGICAGSAFPRAARSNGHTTGTNPPTAGTVDRLT

SEQ ID NO: 14 - protein, 681 aa, Streptomyces cattleya
MTVPAARSGRKVYFIGLNAVPFLPLVAGLLRTYAEQDPRVAAGYDFQEPVFLVDGVQEMAAGITDPDVLALSCYVWNFRRQMKVARLVKERHPGMLVVAGGPHVPDRPGDFFARHPYVDVLVHGEGETAFRELLIERLADHPDYTRVPGVSVRHGTEAVPGRPAERLPRRIETPSPYLLGVMDGAVATCRQRDLRFYALWETNRGCPYSCAFCDWGSATMSALRLFDAERLQEEIEWFAEHDVEDLFVCDANFGILPRDLEIARALADARAATGAPKLIRVNFAKNSNDRVFEISKTWHDADLLMGTTLSMQSTDLDVLEAIDRKNIGLENYRKLQQRYATENIHTYTELILGLPLESPRSFRDGIGSLLEAGNHEDIRVYEFGILPNAPINTPEKIAGYGLRTVPKRLYVEEPGTPDDEAETVDMVMETNAMSRDEWVDCFGFVQAVQFLHNGCYTRYLSMYLRQRHDIGYTAFYERLQQYFGARPDTVLGSIYLRMRKLYHDYIDIPELPLANLVASQPDMAADLARYGKRRGWTIDNWGWLRIANDFERTYTELRDFVAALGVDDTSDPDLAEVFRFQQDVMLRPDYDPAAGRTTEYTADWPGYFTTGELRRRPVRMRLADQRFGANGRYTPVPGDLKAFARAAIGPSYPVSRIGHYRHRFEAAEVTSPAEPVLTEQR

SEQ ID NO: 15 - protein, 474 aa, Streptomyces cattleya
MRVLLVWPRNERALLSDRLSCCEPLPLEYLAGALRGAHDVTIHDLRVDTSLEEYAATHEAPDLIGVAIPYTTSVRVSRDVTHQARRLWPGTPIVLGGHHPTVSAEWLTGFAADWIVAGEGGGPLAHLAAELEAGRTPAPVRGLAPYDARTGLERDRRPKPSALDDLPMPDRTRLAHHRGRYFHSIYRPVALIRFTAGCPYTCKFCSLWRMTDRRYLVKDIDRVLAEIADIDGDNLYVVDDEAFIQPVRMLELADAIDKAGFRKKFHMYVRTDTALRRPDVIARWAEIGLDSVLVGAESMTDEELTGYRKGTDPGQTRRALDLFHGNGVKVRANFIVQPDWSEADFARLGRTVDELQVDMPSFSVLTPLPGTDLYDEAKLGLISDNPELFDCYHSLFRTRLPLERFYGELADLLAGASARTAPGVTGESDPSVFYYSDDDAFDQMLRELRRGADWSHPRESWSPTREPETVAGTG

SEQ ID NO: 16 - protein, 458 aa, Streptomyces cattleya
MAGFLLRCRPEGAVPQHLGAGTGLATEVTRDGADVLVVHGDPLTPGGQAPIHAPLDELIAWTVRAYSRFCAVLVRDDRAVLWSDNGATCPLHYARGADDTLLVATSAGALLPLLGTAPVLGEGDRAVLPGGRFAGVTAVPAGTAVTLAVAGFDTEPLLTRRYHRLPARPTETDPERAVTAVRDALTHAVGRLATGLTEAGVMLSGGVDSSSVAALAAREVTSLSTYTVGTPFGDEFAQAAWFAERLGSKHHELVFEPEQLTALLPEMIRSLETWDLLTLQIAAPACFLLGHIATGARQVMLSGYGADLIFAGLGGTGTETRIERSVAAQTAATAVSNEFNPAYADARDVVVRYPYWTREVMSTALGIRGRLKVRPDTAKWVLRSAVTGILPDEVAWRPKRGIHEGTAMSRMFAAALGSDDRHTQARRLYEMATEVFRDTAAGTDELEGSDEGLAGVAS

SEQ ID NO: 17 - protein, 367 aa, Streptomyces cattleya
MPSPSKLRPITDEDVRRAVRLHFDPQDGTPYWLERDRRDGTDALRKVHGMLDAQQLIGLRDGPDQAHFEEASRRTPLANFVPRRVLTEGGPLWAAQTGGTTGPAKHGTWGARYWADILEFSDEFLDLHGVPRGVDWLFVGPMGPHTTGRLVVAFAERRGGMCFSVDLDPRIVKIFGEEGMTAAYDRYVQHIWDQVAEVARAQRIGVLFCTSRLLEMLPERLGTEALPGLRAIVHAGTTMEPESHRQLREDFFPGVPVVGMYGTSTTGISWQKPFEPEDDHHVVYVPCQPHIALDAVDDDGNEVPFGEEGRVRVWRLTDDALLPGFLERDRARRVQPYGAAAERYPWPWLGDPYSPEFTEGRRIEGVY

SEQ ID NO: 18 - protein, 472 aa, Streptomyces cattleya
VTTVTTGHAGPQRAGAVLDVPAYAGRRPVMSSRPAALTGTTGETVARVGAAGRLVQFQMAQEAAAVARRLRATDDDAWFALLRRAADTLTARVADGTAQPWLTALSAASGLPPRRAARGIETVAADLARMDEILAAQSPDGTVAAYRTGGHHPRWSWRPAGRSVLVKVADNFPTINIEWLQALAARRPVLLSTSRHDPFTPVLLTEALYEAGLPEHAVSVVHGDAPALRRLADQVLWPGEDVPADLPPGKAKTYHFGRSRAVVGAGAADDAWPRLARAAFAGCGRLCTNVSSVIALGDARQAADRLAEEFATRPVLPLDDPAATVPAFPDRARRDALATRIEREIAAGAVDVTEAVTGVPLRVEVDGAAFLRPTVLLVDPGSPLFGTELPFPFTAVAHVPRSKAVAACAGSLIVSVLGDAAGLLGALAEEPGVDKVFGAEEYDRGYHPCDPHEGYLADFLFRKKAVIPGAAG

SEQ ID NO: 19 - protein, 484 aa, Streptomyces cattleya
MQVLLVNARRSPYSGESISAPQLGLLSLASVLREGTFHDTAGTDVHFIDDQLFVLQRPLSTPSEFLRGYRPDIVGIQVLTSSLKNGIKLASEVRHRHPNALTVLGGVGASPIARKLIEENAADVVVRGEGEYSFSQLVHEFGKNGRKNFAKVRGITFRDDEGEVVETPAAPQVVNLDKLPKPARDLADLDLYRRISRGRSGNLVTSRGCSYACAYCYSKHQWGVGQRRHSAARVVDEIRELVEVYGFDRIRIEDDDFVEDVPRMQELCDGIAKSGLQGKFEWEAKARPDLINDDMARMLRESGCFRLLVGVETLSWNLLKRLGRPVKVDVTERAISCLTNNGIGVQATMILGIPGETDEAMRTTITWLQARLGKNKHDLVSPCFFVPFHQEVEKDMAKRVDFTVETSDTDCYTGHIPVTSSMGASIDELWKLYDDMTPSRSQGQYKRIAFLANLQTVQTRLGLLDGDGASNLSNEPAALVPEVQ

SEQ ID NO: 20 - protein, 259 aa, Streptomyces cattleya
MTASHDTADATDRTVGSTSGERAAFARDGVVRWGRLLTPDQIDALRSSVERAFFRDGHPADGVRDLSERQGRPLDLALLHKINLWRTDEACAAQVARADLADRAEALLGGPVRLYRDHVFYKPPGKGDRSRMVLHQDNRYWHLDPPEAITVWMALDDATVENGCVHYVLGSHRHGRVEHVRPEEGAVMIEARTEQEPVAYPAPAGDALVHSVNTLHGSGPNLSDGPRRAYVVVYVRDGVTMRGEPMTSFPLVGDLVRGG

SEQ ID NO: 21 - protein, 240 aa, Streptomyces cattleya
MADGPGAYADPVDLDRVYPRHRTDVPTWLTGLARQAADDSPPELVRYLLDDPARGRPSAVLVLFGTGDAGTDLLLVRRSRTLRSHPDEVCFPGGSVSAGDRDVVHTALRESAEETGLDPAGVAVAGTLRPLRIAWTDFRVTPVLGWWGAEAEPRGDRGEVVSVHRVPLAEFADPANRFRVRYQDGYISPGFLVRELFVWGFTGSIVDWLMRLAGWGRPWDTSRIEELPDALRRYGGGFGR

SEQ ID NO: 22 - protein, 329 aa, Streptomyces cattleya
MGSHRLLRARAGTAGAGPSRRGLIGTSLAGISGLAAGFGSLGARPAAAARPGPVGGTTRLRWFGTNAWEIGFGGRTVLVDPWLTRFTAQRPDGRVDPDTPLTVDHSAVDRHLRAADLILLTHGHYDHIGDLPYVMRKFPSAPVVATETHAHLLTAMGAPTDRVIWARGGEYLDFGDFAVRVLPSLHSMGPDHRYFAPGTLTAPPRRARTVGDLLEGGTLAYHITSDSGASVVNIGTANVIERELTGLRPHVAIVAVPPCGATHRYLERVLTALGHPPHVIPTHHDQLDTPLFRPASVDPGQMRSFRDQVRALGPGCAVTEPQYCDAFTL

SEQ ID NO: 23 - protein, 399 aa, Streptomyces cattleya
VDPEAGSRARGLKGVHRVSANSGGIGGVPGPHNGLTDVPGVRVGHAGRTGDGWLTGVTVVLAPPGGAVAAVDVRGGGPGTRETDALDPRNLVQTIDAVVLTGGSAFGLDAAGGVAAWLEEQGRGFPVGADPSQVVPVVPAAALFDLGRGGTWRARPDAALGRAAVEAAAARPEGDPVEQGGVGAGTGAVVGGLKGGIGTASVVLDSGATVAALAAVNAAGSAVDPATGVLYGARTGLPGEFAGYGVPDAIGADTHARARARLAEAAEETARRRAGGAATLNTTLAVVATDATLTRAQAQKLAGTAHDGLARAVRPVHLLSDGDTVFALSTGRRPLLPDRPDATAARAFGVHLEAGALNEVLAAGADVLTRAVVHAVLAATGVDTPGGVHPSYRELYARP

SEQ ID NO: 24 - protein, 268 aa, Streptomyces cattleya
MDADDCWARAGTVRIRLLGPVELACGTRPVPVTGRRQLRVVAALALEAGRVLSTAGLIASLWADEPPRTAARQLQTSVWMIRRALASVGAPQCVVRSTPAGYLLDPAHYELDSDRFRHAVLTARELQRDGRLAQARARVDEGLALWRGPALGAAAGAGLQPRARRLEEERVFALEQRAGLDLALGRHETAIGELLDLIAQHPLREAAYADLMLALYRSGRQSDALAVYRRAQRVLADELAVRPGPRLAGLERAILRQDESLLAGAAVP

SEQ ID NO: 25 - protein, 137 aa, Streptomyces cattleya
MTPHDSASPTATVRLDHTAVYASDRYLSAEFIAAILGLEVGKPFGPFLPVDLGNGVTLDYYEKRDEPIQSQHYAFIVPEDRFDAVIDRLETVGVTYYADPAHTEPGRVNGLFGGRGAYFADPDGHNMEVMTRPYVRP

SEQ ID NO: 26 - protein, 180 aa, Streptomyces cattleya
VRRVSPAWSGVRGRHVRSAVPARRRSPVTSSAGLPVLSSLDDLARLADGSRGLYVRWSRGPRYDLRETGSADSLTGISLPGLSANPLDVDDWWAGRPLRLWVARRLYDYCHLRHDIGPGVRPWVLRGREVGRGPDNEPLLTDVRPIAWIDPEVIHEARDEVARQRGDWGPLRRPPQASGG

SEQ ID NO: 27 - protein, 62 aa, Streptomyces cattleya
MNTLAWWLRLTVLAVAGGAVALLVQHWTDGRPWPEAGVPAALLVVVALLMGVVSRRSRRDLW

SEQ ID NO: 28 - protein, 253 aa, Streptomyces cattleya
MTNRTAVLSDIHGVLPALEAVLAEPEVRAADRVVLTGDIACGPQPAEVLDLLTALGDRVTWVAGNADRELVEFRRGVRETIPDPIGPWAARQLRPDHLELLASLPLSVRLPVAGLGTVLFCHATPRDDEEVVVVDSRPDRWREVFDGLGPDVDAVICGHTHMPFVRLAHGRLVVNPGSVGMPYGRSGAHWALLGPGVDLRRTPYDTDAAIARLTRDCGYPAIAEWADYYVRARATDTEALAAFGPRDGRGAEG

SEQ ID NO: 29 - protein, 61 aa, Streptomyces cattleya
VPCAPVGPRTPAPPTIWPAPRGPRATRRGGTATSPRCSSTICSAVTWCAARYGSVHQFRLG

SEQ ID NO: 30 - protein, 143 aa, Streptomyces cattleya
MPESIEDPFTPPSPEQARAERVATSLFRIAERHAATEERRRRQTHPYVLAPHEAVRLVAFLLSGAAQHEEDEPEVDRADLLAALTLLPAARADLDEIEAGLLKMARGRGLTWPEIAFGLGLGTPQAARQRYERLSGRISADPE

SEQ ID NO: 31 - protein, 545 aa, Streptomyces cattleya
MFHRISRPGRGLRDGAGTVTRGRRHRPVRGPGERSRCSRPASALAVALTLALTAGVVSPAIAATTPRAAPSAAPPPPAPTTAAGDVLVNGWGDGAGYHLDVATGSGGYDWRELAVLRPAGIDDSSWTGYQCVSGDGRYAAVAILPASAVNLAVARDHGAFAYSVDLRSGAVKPVAAGVALKYHTPGCGTGDTAEFTIDPGNDQRATQVLSADLPSGRLTHVTTVPGQVTSVVPAGDGPVGVLGGELVRIPEDGGRTARPVALASVGGLAYDLRPAAGGGVDFAVQRAAGRSSLIVRERSGHLTTLGEGTHTGLRLMQGRAGHALALEATRLVPGSGVRAVATRGLPGAATGVSLDGGAVLGLGARTAASAPLVLATRTGKVLKRSAAPSARRPDTVLPVPPPGAFGGAAGGLTPSSAGVRGTVSPLTATPKCAVGRNEENRQVMQPGTAQVSWAVQMAEQGLLTGSAYQRPANFANLGLVAYSPNGDFGKVALHHPSGDSWDSVPRSVYQAIVAQESNYSQASWHSLPGIPGNPLIADYYGAGGS

SEQ ID NO: 32 - DNA, 30 bp, Artificial sequence
  Description of Artificial Sequence: degenerated oligonucleotide primer
atcgtctagacsgasacstcsaacgagtts

SEQ ID NO: 33 - DNA, 28 bp, Artificial sequence
  Description of Artificial Sequence: degenerated oligonucleotide primer
atcgaagcttsgasccctcgtggacgcc

(19) United States
(12) Patent Application Publication
(10) Pub. No.: US 2004/0038250 A1
(43) Pub. Date: Feb. 26, 2004

(54) Gene cluster for thienamycin biosynthesis, genetic manipulation and utility

(75) Inventors: Luz Elena Nunez, Gijon (ES); Gloria Blanco, Gijon (ES); Alfredo Brana, Gijon (ES); Carmen Mendez, Oviedo (ES); Jose Antonio Salas, Oviedo (ES)

Correspondence Address: MICHAELSON AND WALLACE, PARKWAY 109 OFFICE CENTER, 328 NEWMAN SPRINGS RD, P O BOX 8489, RED BANK, NJ, 07701

(73) Assignees: ASTUR-PHARMA, S.A.; UNIVERSIDAD DE OVIEDO

(21) Appl. No.: 10/374,903

(22) Filed: Feb. 26, 2003

Related U.S. Application Data
(60) Provisional application No. 60/370,012, filed on Apr. 4, 2002.

(51) Int. Cl. C12Q 1/68; C12N 15/76; C12N 1/21

(52) U.S. Cl. 435/6; 435/486; 435/252.3

(57) ABSTRACT

Isolation, cloning and sequencing of the cluster of genes involved in the biosynthesis of the carbapenem thienamycin by streptomyces cattleya, and the use of those genes to increase thienamycin production, and/or related antibiotics, in the producing strains, and obtaining new derivatives by means of genetic manipulation which implies gene expression, mutagenesis by gene replacement and combinatorial biosynthesis.

### SUMMARY OF THE INVENTION

The present invention relates to the cloning and sequencing of the thienamycin biosynthetic gene cluster from Streptomyces cattleya and the use of the genes included therein to increase yields of thienamycin, or related antibiotics, in the producer strain/s and to obtain novel derivative compounds by genetic manipulation concerning gene expression, mutagenesis by gene inactivation and combinatorial biosynthesis.

### FIELD OF THE INVENTION

The present invention concerns the isolation and identification of a gene cluster involved in the biosynthesis of the carbapenem antibiotic thienamycin by Streptomyces cattleya and provides a tool for the genetic manipulation of the cluster in order to increase thienamycin production and to obtain novel derivatives with improved properties.

### BACKGROUND OF THE INVENTION

Thienamycin (FIG. 1) is the first β-lactam antibiotic of the carbapenem family that was isolated (Kahan et al., J. Antibiot. v. 23: 1255-1265, 1979). It is one of the most potent and broadest in activity spectrum of all known antibiotics and it has an important clinical use in the treatment of infectious diseases, particularly in hospitals. Thienamycin is active against Gram-positive and Gram-negative pathogenic bacteria, both aerobic and anaerobic (including clinical isolates resistant to classic β-lactam antibiotics) and it is highly resistant to bacterial β-lactamases. It is produced at low level by the wild type strain Streptomyces cattleya NRRL 8057, that also produces the convenctional β-lactam antibiotic cephamycin C. Thienamycin is highly unstable and a most stable derivative, named imipenem (N-formidoil thienamycin), is the antibiotic of choice for clinical usage.

The development of recombinant DNA technology has provided a powerful tool that has contributed to the knowledge of gene clusters involved in antibiotic biosynthesis in many actinomycete species. This technology can now be applied to increase yields of antibiotics in the producer strains and to obtain novel derivative compounds with improved properties by expression of genes from selected biosynthetic pathways through combinatorial biosynthesis. Recombinant DNA technology has made possible the isolation of complete antibiotic biosynthetic gene clusters, using among other screening strategies, the screening of gene libraries with DNA probes. This approach relies on the existence of previous genetic information concerning the pathway or related pathways that could allow the construction of a probe using information from a partial aminoacid sequence of a biosynthetic enzyme.

Biosynthesis of classic β-lactam antibiotics, as penicillins, cephalosporins and cephamycins is a well known process that takes place by condensation of L-α-aminoadipic acid, L-cysteine and L-valine to form the tripeptide δ-(L-α-aminoadipyl)-L-cysteinyl-D-valine (ACV) by a non-ribosomal peptide synthetase, named ACVS (encoded by the pcbAB gene). Cyclization of this tripeptide is then carried out by isopenicillin N synthase o IPNS (encoded by the pcbC gene). These two steps are common in all producers of convectional β-lactam antibiotics, both bacteria and fungi, and the corresponding genes and enzymes are very well known and are conserved among all producers despite their phylogenetic origin.

On the other hand, there is biochemical evidence supporting that thienamycin biosynthesis in Streptomyces cattleya could proceed through an alternative pathway to the classic β-lactam antibiotic cephamycin C, also produced by this strain (Williamson et al., J. Biol. Chem. v. 260: 4637-4647, 1985). The bicyclic ring in thienamycin derives from acetate β-lactam carbons) and glutamate (pyrrolidine ring) and it has been proposed that it is formed after condensation of acetyl-S-CoA with y-glutamylphosphate instead of the ACV tripeptide formation as occurs in penicillins, cephalosporins and cephamycins biosynthesis (Williamson et al., 1985, supra). From the genetic point of view, there are no previous reports in the literature concerning the sequencing of the thienamycin biosynthetic gene cluster or any other carbapenem antibiotic produced by Streptomyces species.

A novel mechanism for the biosynthesis of the β-lactam ring, has been reported lately for the biosynthesis of the non classic β-lactams, carbapenems and clavams. These two groups of β-lactam compounds are synthesized by a mechanism never reported in classic β-lactam antibiotic biosynthesis. This alternative mechanism for β-lactam ring biosynthesis involves a novel biosynthetic enzyme, a β-lactam synthetase, that was found to participate first in the biosynthesis of the carbapenem antibiotic (1-carbapen-2-em-3-carboxylic acid) produced by the Gram-negative bacterium Erwinia carotovora (now Pectobacterium carotovorum), from where the gene cluster was cloned (McGowan et al., Mol Microbiol v. 22: 415-426, 1996; Salmond et al. U.S. Pat. No. US0,058,719,22A, 1999). The same mechanism, involving a β-lactam synthetase, was found to be involved in the biosynthesis of the clavam clavulanic acid and forms part of the gene cluster for this β-lactamase inhibitor in the Gram-positive bacterium Streptomyces clavuligerus (Bachmann et al., Proc Nati Acad Sci USA v. 95: 9082-9086, 1998).

The present invention is directed to the aim of the cloning and sequencing of the thienamycin biosynthetic gene cluster from the producing strain Streptomyces cattleya NRRL 8057. The genetic information available about the biosynthetic enzyme, β-lactam synthetase, has been used to design synthetic oligonucleotides and generate a probe that allowed the cloning of the thienamycin gene cluster from Streptomyces cattleya. This is the first instance wherein the gene cluster for producing thienamycin has been isolated. The invention provides as well a tool for the genetic manipulation of the cluster in order to increase thienamycin production and to obtain novel potential derivatives with improved properties.

### BRIEF DESCRIPTION OF THE DRAWINGS

FIG. 1. Structure of the carbapenem antibiotic thienamycin.

FIG. 2. Diagram showing a schematic representation of the restriction endonuclease map of the gene cluster for thienamycin biosynthesis of Streptomyces cattleya NRRL 8057.

FIG. 3. Schematic representation of inserts included in cosmid cosCAT25 and in plasmids pLE14 and pLE22.

FIG. 4. Insertional inactivation of ORF6 by gene replacement.

(A) Construction of the plasmids pHZLE6R and pHZLE6F to be used in the gene inactivation.

(B) Gene replacement of the Streptomyces cattleya wild type strain by insertion of an apramycin resistance cassette in ORF6.

FIG. 5. HPLC analysis:

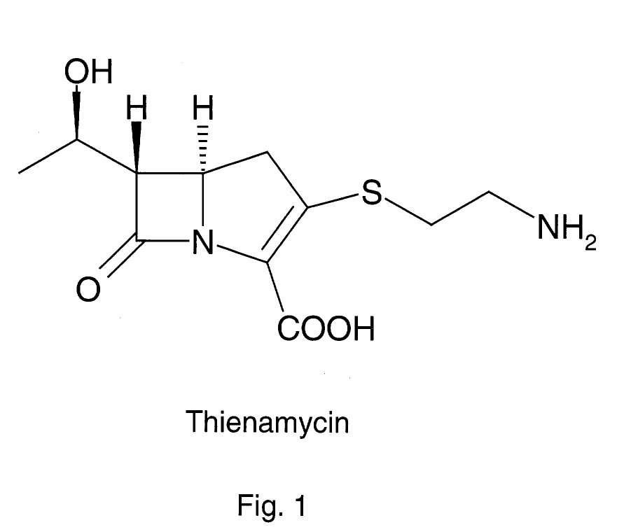

(A) Thienamycin production in the wild type strain of Streptomyces cattleya, with indication of the thienamycin absorption spectrum.

(B) Thienamycin non producing mutant obtained after inactivation of ORF6 by gene replacement using pHZLE6F.

(C) Thienamycin non producing mutant obtained after inactivation of ORF6 by gene replacement using pHZLE6R.

### DETAILED DESCRIPTION OF THE INVENTION

This invention relates to the cloning and sequencing of the gene cluster encoding a β-lactam synthetase involved in the biosynthesis of the carbapenem antibiotic thienamycin. The invention thus relates to novel genes and nucleic acid molecules encoding proteins/polypeptides exhibiting functional activities involved in thienamycin biosynthesis, such proteins/polypeptides themselves, and their uses both in increasing thienamycin production in Streptomyces cattleya and in the generation of novel thienamycin derivatives.

The experimental procedures of the present invention include molecular biology methods conventional in the art. Detailed description of the techniques not explained here are given in the manuals by Hopwood et al. “Genetic manipulation of Streptomyces: a laboratory manual”. The John Innes Foundation, Norwich (1985); by Sambrook et al. “Molecular cloning: a laboratory manual” (1989) and by Kieser et al. “Practical Streptomyces genetics”. The John Innes Foundation, Norwich (2000).

In order to clone the thienamycin biosynthetic gene cluster, a chromosomal DNA cosmid library from Streptomyces cattleya NRRL 8057 was constructed in Escherichia coli, using the bifunctional cosmid pKC505.

For the isolation of the thienamycin biosynthetic gene cluster we have used information concerning a novel biosynthetic enzyme, β-lactam synthetase, above mentioned, to obtain a genetic probe. The strategy was based in the design of a pair of degenerated oligonucleotides according to the conserved regions between the two available β-lactam synthetase sequences (Bachmann et al., 1998, supra and McGowan et al., 1996, supra) as were deduced from the protein alignment. The synthetic oligonucleotides were BLS1 (5′ ATCGTCTAGACSGASACSTCSAACGAGTTS-3′) and BLS4 (5′-ATCGMGCTTSGASCCCTCGTGGACGCC-3′) and were used in PCR-assisted amplification to obtain a probe from the S. cattleya chromosome.

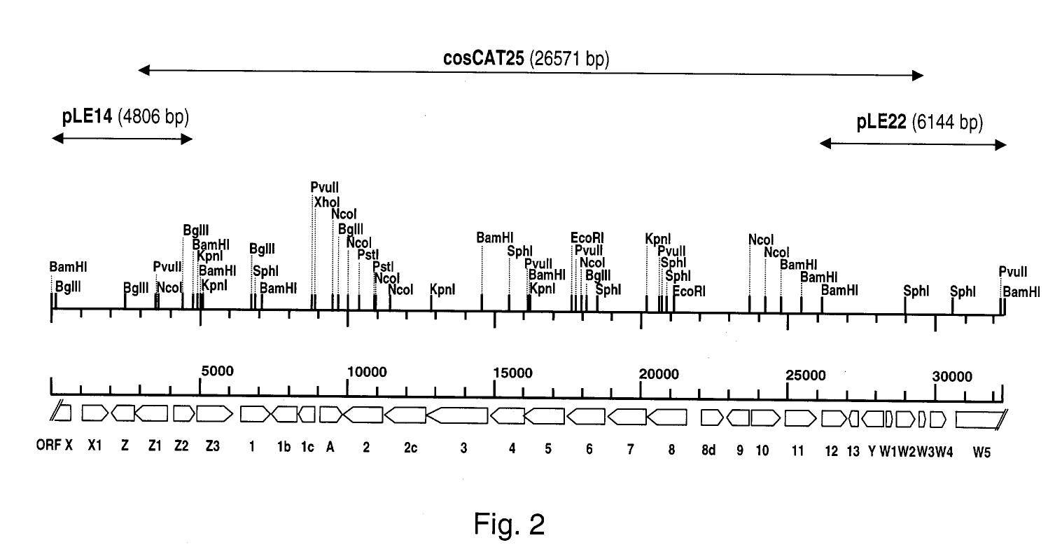

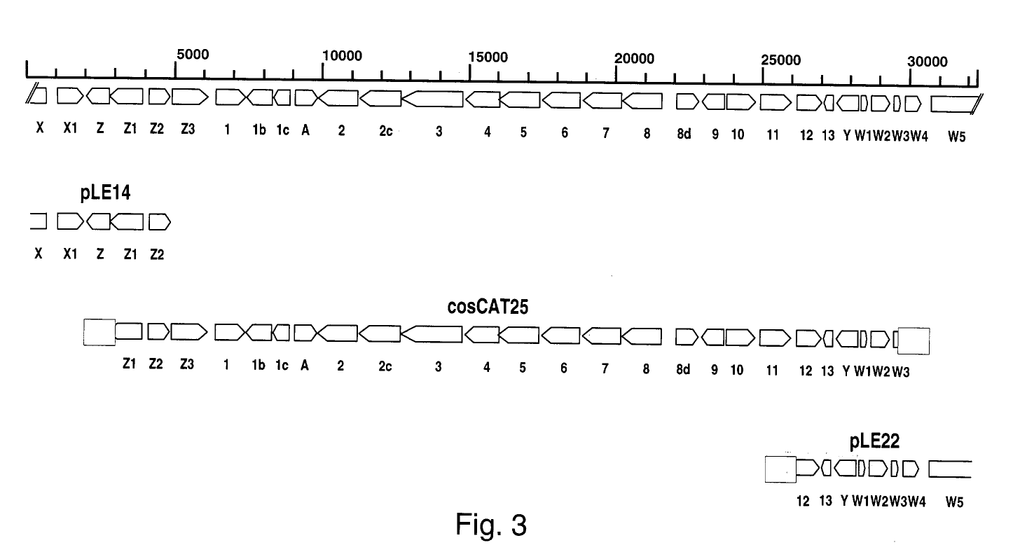

Three cosmid clones, called cosCAT25, cosCAT22 and cosCAT14, were isolated by hibridization with the amplified probe. One cosmid, cosCAT25 (FIG. 2 and 3), was presumed to contain most of the thienamycin gene cluster and was selected for sequencing. In addition two overlapping clones, pLE22 and pLE14, (obtained by subcloning adjacent BamHI fragments from the cosCAT22 and cosCAT14) were further sequenced. Analysis of the nucleotide sequence revealed the presence of 28 complete open reading frames (ORFs) and two incomplete ORFs (FIG. 2 and 3), most of them probably involved in thienamycin biosynthesis. The functions of the genes were concluded after comparison of the deduced amino acid sequences with known sequences available in the data bases and will be described herein below. Some of them would encode structural biosynthetic enzymes, transcriptional activators, proteins involved in exportation, quorum sensing, etc. Among the ORFs coding for structural functions it has been found ORF5, whose deduced product is highly homologous to the β-lactam synthetase proteins that were used in the design of the probe (Bachmann et al., 1998, supra and MacGowan et al., 1996, supra). Two ORFs, (ORF2 and ORF12) would encode regulatory proteins, in fact transcriptional activators highly homologous to ClaR and CcaR from Streptomyces clavuligerus. ClaR works as a transcriptional activator of clavulanic acid biosynthetic genes (Pérez LLarena et al., J. Bacteriol. v. 179: 2053-2059, 1997; U.S. Pat. No. US0,058,210,77A, 1998) and CcaR activates transcription of both clavulanic acid and cephamycin C biosynthetic genes (Paradkar et al., J. Bacteriol. v. 178:6266-6274, 1998).

The involvement of this gene cluster in thienamycin biosynthesis has been demostrated by insertional inactivation of one of the ORFs in the middle of the cluster (ORF6 which forms part of the same transcriptional unit than the β-lactam synthetase homologue), through the insertion of an apramycin resistance cassette generating a thienamycin non producing mutant, as was determined by bioassay and HPLC analysis. The strategy followed for this process has been a gene replacement experiment in which the introduction of plasmid DNA into Streptomyces cattleya was achieved by using intergeneric conjugation from Escherichia coli, according to the method from Mazodier et al., J. Bacteriol. v. 171: 3583-3585 (1989).

The present invention includes a method for increasing the thienamycin producing ability of Streptomyces cattleya. It consists in the overexpression of either (1) regulatory genes from the thienamycin gene cluster, capable of activating gene expression of the cluster, or (2) structural biosynthetic genes, preferably coding for a product that is rate-limiting in the biosynthetic pathway.

The invention further comprises several procedures for manipulating the biosynthetic genes in order to obtain novel thienamycin derivatives: (1) by gene replacement techniques generating mutants in the late steps in thienamycin biosynthesis which could lead to the accumulation of thienamycin intermediates, and (2) by expression of different set of genes in heterologous hosts (β-lactam producers or non producers) in combinatorial biosynthesis experiments.

The present invention will be described in more detailed and illustrated herein below through the following non limiting examples.

### EXAMPLE 1

Cloning of the Gene Cluster for Thienamycin Biosynthesis

1.1. Bacterial Strains, Plasmids and Growth Conditions

Bacterial strains and plasmids used in this study are listed in Table 1. S. cattleya NRRL 8057 was cultured for sporulation on solid Bennet medium (Locci et al. J. of Microbiol., v. 17: 1-60, 1969); for antibiotic production was cultured in liquid R5A medium (Fernández et al, J Bacteriol, v. 180: 4929-4937, 1998) using an inoculum previously grown in liquid TSB medium (Merck). Intergeneric conjugation from E. coli ET12567 (pUB307) into S. cattleya was done according to Mazodier et al. (1989), supra and Flett et al., FEMS Microbial Lett., v. 155: 223-229 (1997). E. coli strains were grown and transformed as described in Sambrook et al. (1989), supra.

TABLE 1
Bacterial strains and Plasmids used in this study

| Strain, plasmid | Properties | Source or reference |
|---|---|---|
| E. coli DH10B | general cloning host | Gibco |
| E. coil ED8767 | host for the cosmid | Sambrook et al. 1987, |
| | gene library | supra |
| E. coil ET12567 | strain for intergeneric | MacNeil et al, Gene, |
| | conjugation | v. 111:61-68, 1992 |
| S. aureus | thienamycin sensitive | ATCC |
| ATCC 6538P | | |
| S. cattleya | wild type, thienamycin | Kahan et al, 1979, |
| NRRL 8057 | producer | supra |
| pBluescript SK | E.coli cloning vector | Stratagene |
| pKC505 | cosmid for gene | Richardson et al., Gene, |
| | library construction | v. 61:231-241, 1987 |
| pUG18 | E. coil cloning vector | Pharmacia |
| pHZ1358 | cosmid for intergeneric | Sun et al., Microbiology, |
| | conjugation | v.148:361-371, 2002 |
| pUB307 | plasmid for | Bennet et al., MGG, |
| | intergeneric | v. 54:205-211, 1977 |
| | conjugation | |

1.2. Analysis of Thienamycin Production

Thienamycin production was qualitatively assayed by bioassay against the thienamycin sensitive strain Staphylococcus aureus ATCC 6538P (cephamycin C resistant). Thienamycin identification and quantitative analysis was performed by HPLC using a reversed phase column (Symmetry C18, 4.6×250 mm; Waters) with acetonitrile and 0.1% trifluoroacetic acid in water as the mobile phase (5:95), at a flow rate of 1 ml/min. Detection and spectral characterization of peaks were made with a photodiode array detector and Millennium software (Waters), and quantification was done after signal integration at 311 nm.

1.3. DNA Manipulation

Plasmid, and total DNA preparations, endonuclease digestions, ligations, etc. were performed as described previously (Sambrook et al., 1989, supra; Kieser et al., 2000, supra; Hopwood et al., 1985, supra). DNA fragments were isolated from agarose gels using the QUIAquick Gel Extraction Kit from QIAGEN (Hilden, Germany) labelled with the use of the DIG DNA Labelling and Detection Kit from Roche Diagnostics (Manheim, Germany) and used for Southern blot analysis according to the manufacturer's manual. DNA sequencing was performed at QIAGEN GmbH (Germany), and the data were analysed with the GCG software (Devereux et al., Nucleic Acids Res. v. 12: 387-395, 1984).

1.4. PCR-Assisted Amplification and Cloning of a DNA Fragment Encoding Part of a β-Lactam Synthetase from the S. Cattleya NRRL8057 Genome

The strategy developed for the cloning of the thienamycin gene cluster was the genetic homology with previously known clusters corresponding to pathways for β-lactam biosynthesis. Available genetic information concerning a β-lactam synthetase, a novel biochemical mechanism for β-lactam biosynthesis, was used for this purpose. Thus, in order to obtain the DNA encoding the thienamycin biosynthesis genes, a S. cattleya NRRL 8057 cosmid gene library was probed with labelled β-lactam synthetase-encoding DNA.

To construct the DNA probe for the screening, degenerate oligonucleotide primers were designed according to conserved amino acid regions within known β-lactam synthetases, β-Is from S. clavuligerus (Bachmann et al., 1998, supra) and carA from E. carotovora (McGowan et al., 1996, supra). The degenerate primers used for amplification corresponded to the conserved regions between the two available β-lactam synthetase sequences, as deduced from the protein alignment, and were designed according to the codon usage table for Streptomyces (Wright & Bibb, Gene, v. 113: 55-56, 1992). The selection of the regions for oligonucleotide design was done avoiding the conserved regions with the related proteins as asparragine synthetase. The synthetic sense nucleotide primer, BLS1, corresponded to the amino acid sequence Thr Asp (Glu) Thr (Leu) Ser Asn Glu Phe and had the sequence 5′- ATCGTCTAG ACG/C GAG/C ACG/C TCG/C AAC GAG TTG/C-3′ (SEQ ID NO: 32), including an Xbal restriction site for cloning (underlined). The antisense nucleotide primer, BLS4, corresponded to the amino acid sequence Gly Val (IIe) His Glu Gly Ser and had the sequence 5′-ATCGAAGCTT G/CGA G/CCC CTC GTG GAC GCC-3′) (SEQ ID NO: 33), including an HindIII restriction site for cloning (underlined). Total DNA obtained from S. cattleya NRRL 8057 cultured on TSB medium (Tryptone Soya Broth, Oxoid) was isolated as described (Kieser et al., 2000, supra). The total genomic DNA from S. cattleya NRRL 8057 was further used as a template for polymerase chain reaction (PCR)-assisted amplification of the DNA fragment from the genome of this organism with the use of BLS1 and BLS4 oligonucleotide primers. It was assumed that both oligonucleotides would allow the amplification of an internal fragment of the β-lactam synthetase encoding gene and that the resulting PCR product would be of approx 0.5 kb in size. The PCR reaction was carried out in a total volume of 50 μl and the PCR mixture contained 0.1 μg of S. cattleya NRRL 8057 total DNA, 200 pm of each oligonucleotide primer, dNTPs (final concentration of 200 μM), 1×PCR buffer from Taq DNA polymerase and 5U of Taq DNA polymerase (Gibco BRL). The PCR was performed on the MJ Research MiniCycler™ with the following program: 1 cycle of denaturation at 98° C. (5 min), 30 cycles of denaturation/annealing/synthesis at 94° C. (1 min)/65° C. (1min)/72° C. (1 min) and 1 cycle of final extension at 72° C. (5 min). A DNA fragment obtained with this procedure was cloned in the Escherichia coli vector pUC18 (using the Xbal/HindIII restriction sites included in the synthetic oligonucleotides) and was subjected to further DNA sequencing using standard techniques in Molecular Biology. DNA sequence analysis of the resulting amplified fragment followed by conceptual translation and database search revealed that the PCR product encoded a region homologous to part of known β-lactam synthetases (Bachmann et al., 1998, supra and McGowan et al., 1996, supra). However, the amplified DNA fragment was shorter than expected initially. In fact it was of 0.22 kb in size, due to the annealing of the BLS1 oligonucleotide to an internal region of the expected in the initial design. Once confirmed that the amplified DNA fragment encodes part of a β-lactam synthetase it was used as a probe for screening a S. cattleya NRRL 8057 cosmid gene library (see below).

1.5. Construction and Screening of the S. Cattleya NRRL8057 Gene Library

The S. cattleya NRRL 8057 gene library was constructed in the bifunctional cosmid pKC505 (Richardson et al., Gene, v. 61: 231-241, 1987), which is able of replication both in Escherichia coli and Streptomyces. Total DNA from S. cattleya NRRL 8057, isolated as described above, was partially digested with Sau3Al and fragments of about 35 kb were dephosphorylated by alkaline phosphatase treatment (Roche Diagnostics, Mannheim). The cosmid vector was linearized with Hpal, dephosphorylated, and digested with BamHI to generate both cosmid arms. Insert DNAs and vector were ligated and packaged in vitro using a commercial packaging kit from Roche Diagnostics Mannheim. The recombinant phage particles were used to infect E. coli ED8767 and transductants selected on trypticasein-soy agar (TSA) plates (containing 10 μgml⁻¹ tobramycin). Approximately. 3000 transductants were cultured on microtiter plates and, after incubation at 28° C. for 24 h, kept with 25% glycerol at −70° C.

In order to clone the gene cluster for thienamycin biosynthesis the genomic library of S. cattleya NRRL 8057 was screened by in situ colony hybridization with the amplified probe corresponding to an internal fragment of the β-lactam synthetase encoding gene (see above). For the screening of the cosmid library the DNA probe was labelled with α-P³² dCTP and hybridization was carried out using the Rediprime DNA labelling system (Amersham) according to the manufacturer's manual. Three hybridising cosmid clones, cosCAT25, cosCAT22 and cosCAT14, were isolated and selected for further analysis. After Southern blot analysis using the same probe it was determined that the three cosmids show overlapping restriction maps and one of them, cosCAT25 (FIG. 2 and 3), was selected for sequencing analysis. In addition, two overlapping clones, pLE22 and pLE14 (FIG. 2 and 3), obtained after subcloning adjacent BamHI fragments from the cosCAT22 and cosCAT14, were also sequenced.

### EXAMPLE 2

Sequence Analysis of the Gene Cluster for Thienamycin Biosynthesis and the Deduced Functions from the Genes

Sequence analyses were made using the GCG sequence analysis software package (Version 8: Genetics Computer Group, Madison, Wis., USA). The translation table was modified to accept also GTG as a start codon. Codon usage was analyzed using published data (Wright and Bibb, 1992, supra)

Computer-assisted analysis of the DNA sequence (FIG. 2) comprised the region cloned in cosmid cosCAT25 (26571 bp) and the two overlapping BamHI fragments from cosmids cosCAT22 and cosCAT14 that were cloned in pLE14 (4806 bp) and pLE22 (6144 bp) (FIG. 3). According to the CODONPREFERENCE program the sequenced DNA fragment revealed 28 complete open reading frames (ORFs) and two 5′ ends of the other ORFs (ORFX and ORFW5). The functions of the genes were concluded by comparing the amino acid sequences translated from their base sequences to the known sequences in the data banks. The results are shown in Table 2 referring to the sequence data given in the application.

TABLE 2

| | | A- | | |
|---|---|---|---|---|
| | | mino | | |
| Gene | Position | acids | Deduced function | Remarks |
| orfX | −751 | >250 | unknown | not complete |
| | compl | | | SegID.NO:2 |
| orfX1 | 1034-1924 | 296 | unknown | SegID.NO:3 |
| orfZ | 1940-2719 | 259 | putative | SeqID.NO:4 |
| | compl | | oxidoreductase | |
| orfZ1 | 2850-3932 | 360 | putative | SeqIDNO:5 |
| | compl | | efflux pump regulador | |
| orfZ2 | 4117-4755 | 212 | putative | SeqID.NO:6 |
| | | | efflux protein | |
| | | | LysE family | |
| orfZ3 | 4945-6036 | 363 | putative | SegID.NO:7 |
| | | | oxidoreductase | |
| orf1 | 6288-7172 | 294 | enoyl-CoA | SeqID.NO:8 |
| | | | hydratase | |
| | | | CarB homologue | |
| orf1b | 7156-8139 | 327 | unknown | SeqID.NO:9 |
| | compl | | | |
| orf1c | 8136-8927 | 263 | putative | SeqID.NO:10 |
| | compl | | hydroxylase | |
| orfA | 9171-9845 | 224 | putative epoxide | SeqID.NO:11 |
| | | | hydrolase | |
| orf2 | 9767-11197 | 476 | putative transcriptional | SeqlD.NO:12 |
| | compl | | activator ClaR | |
| | | | homologue | |
| orf2c | 11289-12740 | 483 | putative transport | SeqID.NO:13 |
| | compl | | protein | |
| orf3 | 12737-14782 | 681 | putative | SeqID.NO:14 |
| | compl | | methyltransferase | |
| orf4 | 14838-16262 | 474 | putative | SeqID.NO:15 |
| | compl | | methyltransferase | |
| orf5 | 16234-17610 | 458 | putative β-lactam | SeqID.NO:16 |
| | compl | | synthetase | |
| orf6 | 17612-18715 | 367 | unknown | SeqID.NO:17 |
| | compl. | | | |
| orf7 | 18754-20172 | 472 | unknown | SeqID.NO:18 |
| | compl | | | |
| orf8 | 20169-21623 | 484 | putative | SeqID.NO:19 |
| | compl | | methyltransferase | |
| orf8d | 22038-22817 | 259 | unknown | SeqID.NO:20 |
| orf9 | 22912-23634 | 240 | unknown | SeqID.NO:21 |
| | compl | | | |
| orf10 | 23744-24733 | 329 | unknown | SeqID.NO:22 |
| orf11 | 24784-25983 | 399 | unknown | SeqID.NO:23 |
| orf12 | 26146-26952 | 268 | transcriptional | SeqID.NO:24 |
| | | | activator | |
| | | | CcaR homologue | |
| orf13 | 26980-27393 | 137 | unknown | SeqID.NO:25 |
| | compl | | | |
| orfY | 27614-28156 | 180 | unknown | SeqID.NO:26 |
| | compl | | | |
| orfW1 | 28224-28412 | 62 | unknown | SeqID.NO:27 |
| orfW2 | 28485-29246 | 253 | unknown | SeqID.NO:28 |
| orfW3 | 29382-29567 | 61 | unknown | SeqID.NO:29 |
| orfW4 | 29766-30197 | 143 | unknown | SeqID.NO:30 |
| orfW5 | 30695- | >545 | unknown | not complete |
| | | | | SeqID.NO:31 |

### EXAMPLE 3

Insertional Inactivation of the Thienamycin Gene Cluster by Gene Replacement

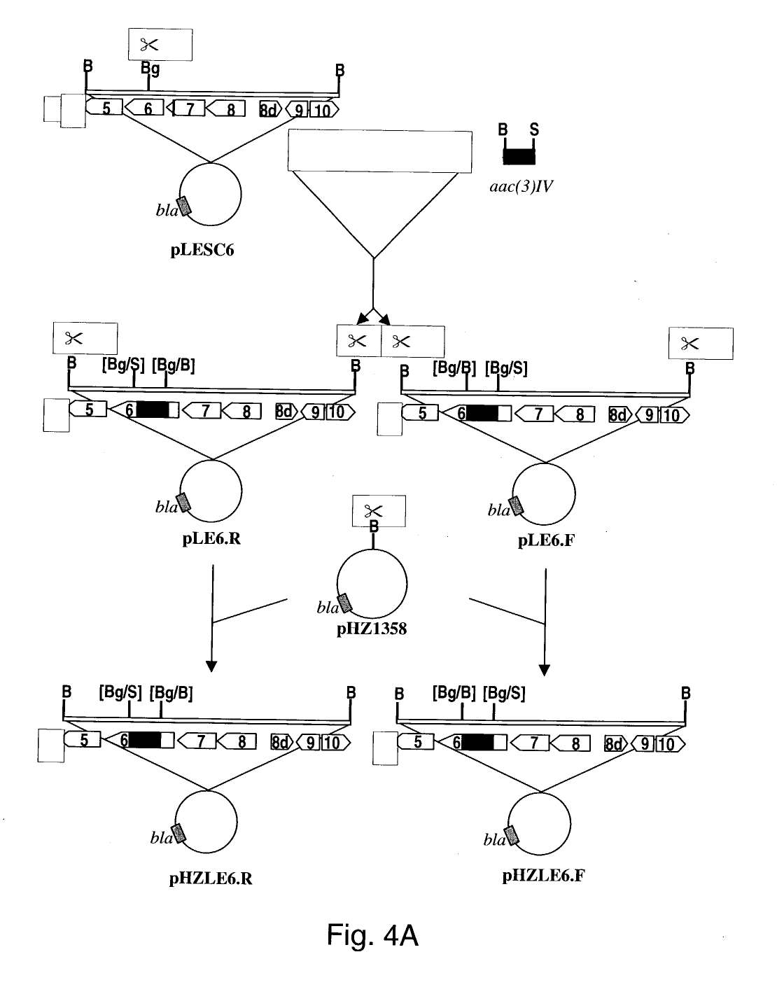

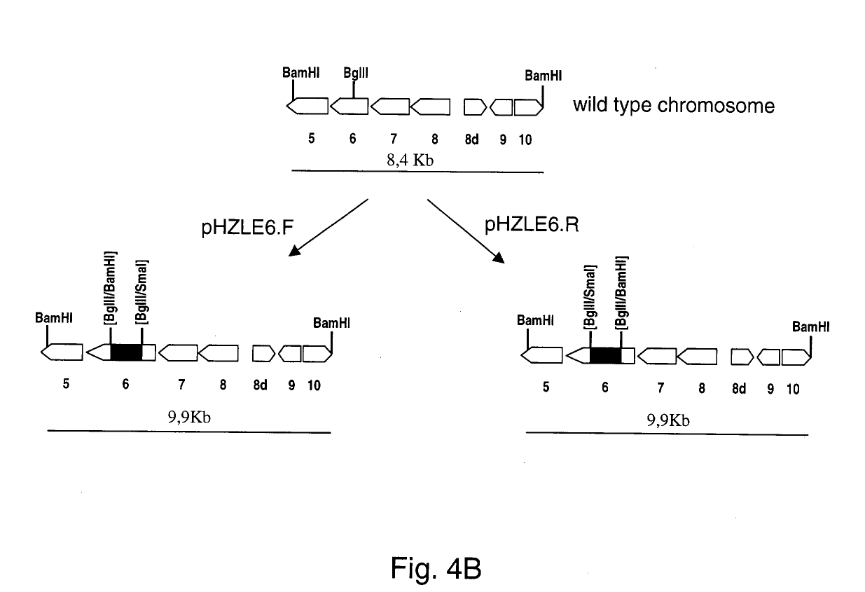

In order to demonstrate the involvement of the cloned gene cluster in thienamycin biosynthesis, ORF6, immediately upstream of the β-lactam synthetase homologue (ORF5) was inactivated by insertion of an apramycin resistance cassette containing the aac(3)IV gene (Stanzak et al. Biotechnology v. 4: 229-232, 1986). For insertional inactivation of chromosomal genes in Streptomyces the gene inactivation is usually created on a suitable vector in E. coli before introducing the construct into Streptomyces for recombination with the chromosome. For this purpose, a 8.4 kb DNA BamHI fragment from cosmid cosCAT25 was first cloned into the pUC18 vector in E. coli, resulting in the plasmid pLESC6 (FIG. 4A). In this later construction, an apramycin resistance cassette was independently inserted in both orientations in the unique Bg/II restriction site (blunt ended) localized in the ORF6 coding region, generating pLE6F (with the apramycin cassette in forward orientation) and pLE6R (with the apramycin cassette in reverse orientation). From these two constructions in which ORF6 was insertional inactivated by the apramycin resistance cassette, the 9.9 kb DNA BamHI fragment was excised and ligated to the conjugative vector pHZ1358, previously digested with the same restriction enzyme, generating pHZLE6F and pHLE6R (FIG. 4A).

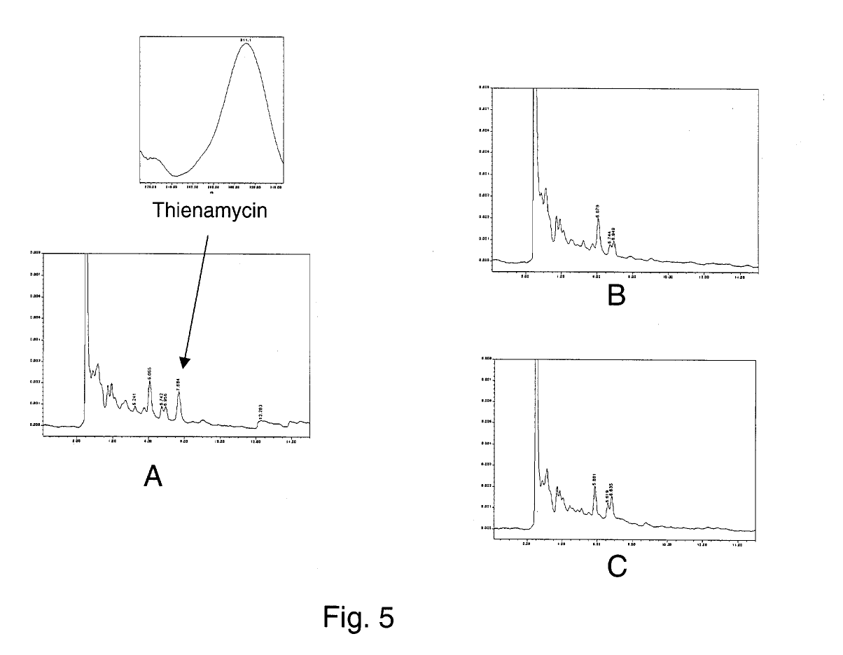

The recombinant plasmids constructed for the gene replacement experiments (pHZLE6F and pHZLE6C) were introduced into S. cattleya by intergenic conjugation from E. coli ET12567 (pUB307) as described by Mazodier et al. (1989), supra. A double crossover is necessary to obtain the replacement of the wild type copy of the gene by the mutated one. The transconjugants in which a double crossover event has happened were selected for apramycin resistance and thiostrepton sensitivity. Replacement in the chromosome of the wild type copy of the gene by the mutated one was confirmed in the transconjugants by Southern blot analysis with the use of labelled 8.4 kb BamHI fragment from plasmid pLESC6 (FIG. 4B). One of each replaced mutant (with apramycin gene inserted in different orientation) was tested for thienamycin production in parallel with the parental strain NRRL 8057 by bioassay and HPLC (see above for methods under “analysis of thienamycin production”). Both mutants, with independence of the apramycin gene orientation, were shown to be non producers of thienamycin (FIG. 5), confirming the involvement of the cluster in thienamycin biosynthesis.

Deposited Microorganisms

| Microorganism | Accession number | Date of deposit |
|---|---|---|
| E. coli ED8767/cosCAT25 | CECT 5877 | March 7^{th} 2002 |
| E. coli DH10B/pLE14 | CECT 5876 | March 7^{th} 2002 |
| E. coli DH10B/pLE22 | CECT 5875 | March 7^{th} 2002 |

1. A procedure for the isolation and purification of a DNA fragment, which contains the gene cluster for the thienamycin biosynthetic pathway of the bacterium Streptomyces cattleya, being included in a 32329 bp fragment of the S. cattleya genome. The process comprises the following steps:
(a) forming a genomic DNA library of a thienamycin producing microorganism;
(b) transfecting clones from said library into host cells;
(c) designing degenerated oligonucleotides for the isolation of the thienamycin biosynthesis gene cluster.
(d) constructing a probe comprising a nucleotide sequence from a thienamycin biosynthesis gene cluster.
(e) hybridising said probe with a genomic DNA library derived from said microbe.
(f) isolating said gene cluster from the positive hybridising clones.

2. The invention provides a nucleic acid molecule according to claim 1, comprising:
(a) a nucleotide sequence as shown in SEQ ID NO: 1; or
(b) a nucleotide sequence which is the complement of SEQ ID NO: 1; or
(c) a nucleotide sequence which is degenerate with SEQ ID NO: 1; or
(d) a nucleotide sequence hybridising under conditions of high stringency to SEQ ID No. 1, to the complement of SEQ ID NO: 1, or to a hybridisation probe derived from SEQ ID NO: 1 or to the complement thereof; or
(e) a nucleotide sequence having at least 80% sequence identity with SED ID NO: 1;
(f) a nucleotide sequence having at least 65% sequence identity with SEQ ID NO: 1 wherein said sequence preferably encodes or is complementary to a sequence encoding at least a thienamycin biosynthetic enzyme or a part thereof.

3. A nucleic acid molecule comprising a part of a nucleotide sequence as defined in claim 1 and claim 2, wherein said part is at least 15 nucleotides in length.

4. A nucleic acid molecule as claimed in any one of claims 2 to 3 which encodes one or more polypetides or comprises one or more genetic elements, having functional activity in the synthesis of a β-lactam antibiotic or a β-lactam precursor.

5. A nucleic acid molecule as claimed in claim 4, wherein said β-lactam antibiotic is thienamycin or a thienamycin precursor.

6. A nucleic acid molecule as claimed in any one of claims 2 to 3 which encodes one or more polypeptides, or comprises one or more genes and/or one or more regulatory sequences, and/or one or more coding or noncoding genetic elements, having functional activity in the synthesis of a β-lactam antibiotic or a β-lactam precursor.

7. A nucleic acid molecule as claimed in claim 6, wherein said β-lactam antibiotic or a β-lactam precursor is thienamycin or a thienamycin precursor.

8. A nucleic acid molecule comprising a nucleotide sequence encoding one or more amino acid sequences selected from SEQ ID Nos: 2 to 31, or a nucleotide sequence which is complementary thereto or degenerate therewith or comprising a nucleotide sequence which encodes one or more amino acid sequences which exhibit at least 60% sequence identity with any one of SEQ ID Nos: 2 to 31.

9. A nucleic acid molecule as claimed in claim 8 which encodes one or more amino acid sequences which exhibit at least 85% sequence identity with any one of SEQ ID Nos: 2 to 31.

10. A polypeptide encoded by a nucleic acid molecule as defined in any one of claims 2 to 9.

11. A polypeptide as claimed in claim 10, comprising:
(a) all or part of an amino acid sequence as shown in any one or more of SEQ ID Nos: 2 to 31; or
(b) all or part of an amino acid sequence which has at least 60% sequence identity with any one or more of SEQ ID Nos: 2 to 31.

12. A polypeptide as claimed in claim 11, wherein said amino acid sequence at (b) has at least 85% sequence identity with any one or more of SEQ ID Nos: 2 to 31.

13. A polypeptide as claimed in any one of claims 10 to 12 having functional activity in the synthesis of a carbapenem antibiotic or β-lactam moiety.

14. A recombinant DNA molecule, which comprises the DNA fragment of any one of the claims 2 to 9, or a part thereof having similar characteristics, cloned in a vector replicating in Streptomyces or in E. coli.

15. The recombinant DNA according to claim 14 which is the cosmid cosCAT25 deposited in E. coli strain ED8767/CAT25 with the accession number CECT 5877.

16. The recombinant DNA according to claim 14 which is the plasmid pLE14 deposited in E. coli strain DH10B/LE14 with the accession number CECT 5876.

17. The recombinant DNA according to claim 14 which is the plasmid pLE22 deposited in E. coli strain DH10B/LE22 with the accession number CECT 5875.

18. A host cell or transgenic organism comprising a nucleic acid molecule as defined in any one of claims 2 to 9.

19. A host cell or transgenic organism comprising a vector as defined in claim 14.

20. Use of the genes derived from the DNA fragment of claims 2 to 9 in the production of β-lactam metabolites.

21. Use of the genes derived from the DNA fragment of claims 2 to 9 in the production of thienamycin, thienamycin derivatives or thienamycin precursors.

22. Use of the genes derived from the DNA fragment of claims 2 to 9 to increase β-lactam metabolites production.

23. Use of the genes derived from the DNA fragment of claims 2 to 9 to increase production of thienamycin, thienamycin derivatives or thienamycin precursors.

24. Use of a DNA molecule, as claimed in any one of the claims 2 to 9, in the inactivation of genes involved in thienamycin biosynthesis.

25. Use of a DNA molecule, as claimed in any one of the claims 2 to 9, in PCR amplification techniques leading to the isolation and/or use of genes involved in thienamycin biosynthesis.

26. Use of host cells or transgenic organisms as claimed in any one of claims 18 to 19 in the production of β-lactam metabolites.

27. Use of host cells or transgenic organisms as claimed in any one of claims 18 to 19 in the production of thienamycin, thienamycin derivatives or thienamycin precursors.

28. A process for increasing β-lactam production in a bacterial host, comprising transferring the DNA fragment of claim 2 to 9 into a Streptomyces host, cultivating the recombinant strain obtained, and isolating the β-lactam produced.

29. The process according to claim 28, wherein the Streptomyces host is a Streptomyces cattleya host.

30. The process according to claim 29, wherein the Streptomyces cattleya host is a mutant strain derived from S. cattleya NRRL 8057.

31. A process according to claims 28 to 30, wherein the β-lactam compound is thienamycin, a thienamycin derivative or a thienamycin precursor.

32. A process for generating thienamycin derivatives or thienamycin precursors by inactivation of genes derived from the DNA fragment of claims 2 to 9.

33. A process as claimed in any one of the claims 20 to 27 for using the thienamycin intermediates or thienamycin derivatives as starting compounds for chemical synthesis of β-lactam products.
